(19) United States
(12) Patent Application Publication
(10) Pub. No.: US 2022/0202742 A1
(43) Pub. Date: Jun. 30, 2022

(54) DOSAGE FORMS FOR PREVENTING DRUG-FACILITATED ASSAULT

(71) Applicant: CLEXIO BIOSCIENCES LTD., Jerusalem (IL)

(72) Inventors: Michal YAHAV, Jersusalem (IL); Avi ROSENBERGER, Jersusalem (IL)

(21) Appl. No.: 17/608,215

(22) PCT Filed: May 7, 2020
(86) PCT No.: PCT/IB2020/054352

§ 371 Date: Nov. 2, 2021

Related U.S. Application Data
(60) Provisional application No. 62/844,645, filed on May 7, 2019.

(51) Int. Cl. A61K 31/135; A61K 9/00

(52) U.S. Cl. CPC A61K 31/135; A61K 9/20; A61K 9/007; A61K 9/0095

(57) ABSTRACT

Disclosed herein are immediate release oral dosage forms that comprising esketamine and one or more excipients that produce a visual indication of the presence of the dosage form within a vessel containing a liquid beverage. The visual indication can assist in thwarting an attempt to use the esketamine to render another individual more susceptible to criminal exploitation.

### CROSS-REFERENCE TO RELATED APPLICATIONS

The present application claims the benefit of priority to U.S. Provisional App. No. 62/844,645, filed May 7, 2019, the entire contents of which are incorporated herein by reference.

### TECHNICAL FIELD

The present disclosure pertains to oral dosage forms that produce a visual indication of the presence of the dosage form within a vessel containing a liquid beverage and contain abuse-deterrent features.

### BACKGROUND

Pharmaceutical products, including both prescription and over-the-counter pharmaceutical products, while useful for improving health or alleviating undesirable symptoms of a person in need, are also susceptible to abuse. In certain instances, “abuse” of an active pharmaceutical ingredient by an individual can include surreptitiously administering the drug to another person in order to render the other person more susceptible to criminal exploitation, such as sexual assault, physical assault, or theft. The exploitation is made easier by altering the intended victim's mental state, e.g., rendering the intended victim confused, unable to defend themselves, or unable to remember. For example, one or more dosage forms containing the drug may be placed into a beverage intended for another individual, and allowing the other individual to consume the beverage, and thereby ingest the drug. When the drug is one that is capable of altering the mental state of the individual, that person can then become more susceptible to criminal exploitation.

Commonly abused active pharmaceutical ingredients include psychoactive drugs, anxiolytics, sedative hypnotics, stimulants, depressants, and analgesics such as narcotic analgesics, among others. These drugs are sometimes referred to as “date rape” drugs or “club drugs.”

Although the pharmaceutical industry has identified of a variety of abuse deterrent (sometimes referred to as “abuse-resistant”) features useful with oral dosage forms, those features generally service to protect against self-harm. That is, those abuse-resistant features reduce the likelihood of self-administered abuse. But there is a need to develop dosage forms that prevent or deter the surreptitious administration of drugs to unknowing victims. In particular, there is a need to develop dosage forms that prevent or deter an unknowing victim from consuming a liquid (e.g., beverage) that has been unadulterated with a drug intended to make the victim confused, unable to defend themselves, or unable to remember.

### SUMMARY

Disclosed herein are enteral dosage forms comprising esketamine and one or more excipients that produce a visual indication of the presence of the dosage form within a vessel containing a liquid beverage, the visual indication including dispersion of visible particles, cloudiness, or both, within the beverage.

Also provided herein are enteral dosage forms comprising an active pharmaceutical agent and a gelling polymer, wherein the dosage form produces a visual indication of its presence within a vessel containing a liquid beverage, the visual indication including dispersion of visible particles, cloudiness, or both, within the beverage.

The present disclosure also provides methods of preventing drug-facilitated criminal exploitation of a subject comprising providing a dosage form as described herein.

The present disclosure also pertains to methods of producing a visual indication of a dosage form's presence within a vessel containing a liquid beverage intended for ingestion by a subject that is a target of drug-facilitated criminal exploitation comprising providing a dosage form as described herein.

### BRIEF DESCRIPTION OF THE DRAWINGS

FIG. 1A, FIG. 1B, and FIG. 1C illustrate embodiments of core-shell particles, in cross section, for inclusion in the presently disclosed dosage forms.

FIG. 2A and FIG. 2B also illustrate embodiments of core-shell particles, in cross section.

FIG. 3 is a perspective view of an embodiment of a dosage form as disclosed herein.

FIG. 4 shows the results of single-tablet dissolution testing of an inventive dosage form according to the present disclosure and of a comparative dosage form.

FIG. 5 provides an image of a dual screen apparatus that was used to test a supratherapeutic dose of an inventive dosage form with gel retained on the screens.

FIG. 6 provides an image of a dual screen apparatus that was used to test a supratherapeutic dose of a comparative dosage form with gel retained on the screens.

FIG. 7 provides an image of a dual screen apparatus that was used to test a supratherapeutic dose of a comparative dosage form with gel retained on the screens.

FIG. 8 provides an image of a gel resulting from a supratherapeutic dose of inventive dosage forms, suspended in a test medium.

FIG. 9 provides an image of a gel resulting from a supratherapeutic dose of comparative dosage forms, suspended in a test medium.

FIG. 10A provides images of the results of placing esketamine, a crushed dosage form according to the present disclosure, and an intact dosage form according to the present disclosure, respectively, into vessels containing a cola beverage.

FIG. 10B provides additional images of the results of placing esketamine, a crushed dosage form according to the present disclosure, and an intact dosage form according to the present disclosure, respectively, into vessels containing a cola beverage.

FIG. 11 provides images of the results of placing esketamine, a crushed dosage form according to the present disclosure, and an intact dosage form according to the present disclosure, respectively, into vessels containing water.

FIG. 12 provides images of the results of placing esketamine, a crushed dosage form according to the present disclosure, and an intact dosage form according to the present disclosure, respectively, into vessels containing white wine.

FIG. 13A provides images of the results of placing esketamine, a crushed dosage form according to the present disclosure, and an intact dosage form according to the present disclosure, respectively, into vessels containing vodka.

FIG. 13B provides additional images of the results of placing esketamine, a crushed dosage form according to the present disclosure, and an intact dosage form according to the present disclosure, respectively, into vessels containing vodka.

### DETAILED DESCRIPTION OF ILLUSTRATIVE EMBODIMENTS

Abuse of an active pharmaceutical ingredient (API) is not limited to the voluntary ingestion, injection, or insufflation of a dosage form or the API from the dosage form by an individual in first-hand possession of the dosage form. An API is also abused, albeit not in the above-noted conventional sense, when a dosage form is used to administer an API to an individual who does not intend to ingest the API, but who is instead an intended target of criminal exploitation. In such instances, administration can include placing one or more of the dosage forms into a beverage or food item of the intended target of criminal exploitation, such that, if the incorporation of the dosage form into the beverage or food item is undetected and the beverage or food item is consumed, the API from the dosage form can alter the mental state of the targeted individual and render them more susceptible to criminal exploitation, such as sexual assault. For example, the targeted individual can be rendered less aware of his or her surroundings. This type of scenario can readily occur in social clubs or party environments where consumption of beverages is frequent, a darkened ambiance renders surreptitious incorporation of a dosage form into a beverage easier, distractions in the form of loud music and social interactions are present, and bathroom visits by an intended target may present opportunities for incorporation of a dosage form into a drink that has been left behind.

The phenomenon of sexual assault (e.g., “date rape”), violent assault, or theft can originate from such circumstances, as an individual who is at least partially incapacitated by unwitting ingestion of an API can be rendered more susceptible to this type of criminal exploitation. However, the present dosage forms are capable of thwarting an attempt to use an API for this purpose, by producing a visual indication of the presence of the dosage form within a vessel containing a liquid beverage, wherein the visual indication is capable of alerting the intended target of the criminal exploitation to the presence of the dosage form in the beverage.

The presently disclosed dosage forms produce a visual indication within a vessel containing a liquid beverage, such as water, a carbonated beverage, a malted beverage, wine, or liquor. As used herein, “water” refers to non-carbonated drinking water, e.g., tap water, purified water, spring water, mineral water, distilled water, and the like. A “carbonated beverage” can refer to sparkling water (seltzer), sweetened soda beverages (such as cola), or diet soda beverages (e.g., diet cola). “Malted beverages” refer to beer or malt liquor. “Wine” refers to red, white, rose, or blush wine that is dry, semi-dry, or sweet. “Liquor” or “spirits” refers to relatively high-alcohol content beverages (typically above 20% alcohol by volume, and as high as 80-90% alcohol by volume, and often around 30-45% alcohol by volume), such as vodka, whisky, scotch, gin, tequila, rum, and the like.

The presently disclosed dosage forms produce a visual indication of the presence of the dosage form within a vessel containing a liquid beverage. In some embodiments, the visual indication includes a dispersion of visible particles, cloudiness, or both, within the beverage. The visual indication may also or alternatively include at least a portion of the dosage form that is buoyant on the surface of the beverage, a film at the surface the beverage, a foam resulting from an effervescent reaction between the dosage from and the beverage, residue from the dosage form on an inner surface of the vessel, or any combination thereof. In some instances, the visual indication includes an absence of a film or foam at an upper surface of the beverage.

In some aspects, the presently disclosed dosage form produce a visual indication of the presence of the dosage form within a vessel via an increase in the turbidity of the beverage, as compared to the beverage prior to the introduction of the dosage form. “Turbidity” refers to the cloudiness or haziness of a beverage caused by the presence of the dosage form, or part thereof, in the beverage. Turbidity can be measured as Nephelometric Turbidity Units (NTU) using a nephelometer, according to EN ISO 7027.

For example, when the beverage is a carbonated beverage or a malted beverage, such as a cola or beer, the visual indication can include, inter alia, a significant effervescent reaction which disintegrates the remaining excipients and mixes them with the beverage. The result of this reaction includes the presence of a strong residue on the inner walls of the vessel containing the beverage, as well as a surface foam. In certain instances, depending on the volume of the beverage relative within the vessel, the visual indication can include a significant effervescent reaction that results in a foamy mixture of the dosage form and the beverage that fills or nearly fills the vessel, and can even overflow to the outside of the vessel to at least some degree. In such instances, as above, the result of this reaction includes the presence of a strong residue on the inner walls of the vessel containing the beverage, as well as a surface foam.

When the beverage is a liquor or spirit, such as vodka, the visual indication can include breaking down of the tablet and the resulting appearance of particles representing well dispersed residue of the dosage form within the beverage.

When the beverage is wine, such white wine, the visual indication can include an increase in turbidity within the beverage, the appearance of a film or foam on the surface of the beverage, or both.

In some embodiments, the dosage forms produce effervescence when introduced into a liquid beverage that can contribute to the elicitation of multiple visual indications, for example, that include one or more of the types of visual indications described above. Without wishing to be bound by any particular theory of operation, the elicitation of one or more visual indications by effervescence from the dosage form may be due to its effect in combination with one or more other excipients, such as a gelling polymer. In this manner, the dosage form can be said to produce an effervescent effect that in turn drives the appearance of one or more visual indications through its combination with one or more excipients, by more efficiently exposing one or more excipients to the liquid beverage, or both.

Abuse deterrent features can also include the ability of a dosage form to produce a highly viscous liquid or gel when crushed and exposed to a solvent, when administered in supratherapeutic doses (in doses that are higher than what is prescribed pursuant to a legitimate treatment regimen), or both, in order to prevent abuse by such methods as intravenous injection, nasal insufflation, and oral supratherapeutic dosing. One challenge associated with incorporating this type of abuse-deterrent feature is whether the resulting viscous liquid or gel possesses characteristics that are required for preventing the above-noted abuse modalities. For example, in order to prevent drug from being released in a dosage form in sufficient quantities to produce the desired effect of the abuse, a gel must be appropriately viscous and uniform. A gel that is runny or non-uniform (e.g., containing portions that are more fluid and portions that are less fluid) will be less effective in blocking commonly-attempted forms of abuse and release of drug. The present disclosure pertains to dosage forms that produce high quality abuse deterrent features when subjected to attempted abuse. In basic terms, the dosage forms that are disclosed herein exhibit an immediate release profile of drug when administered to a human in therapeutic doses, and an extended release profile of drug when administered to a human in supratherapeutic doses.

More particularly, it has been discovered that specific ratios of gelling polymer, particularly carbomer gelling polymer, to pH adjusting compound within the matrix of the dosage forms can be critical for allowing the dosage form, when administered in supratherapeutic doses, to produce a gel having superior physical characteristics for purposes of thwarting abuse of esketamine from the dosage forms. This finding is described more fully, infra.

In the present disclosure, the singular forms “a”, “an”, and “the” include the plural reference, and reference to a particular numerical value includes at least that particular value, unless the context clearly indicates otherwise. Thus, for example, a reference to “a filler” is a reference to one or more of such reagents and equivalents thereof known to those skilled in the art, and so forth. Furthermore, when indicating that a certain element “may be” X, Y, or Z, it is not intended by such usage to exclude in all instances other choices for the element.

When values are expressed as approximations, by use of the antecedent “about”, it will be understood that the particular value forms another embodiment. As used herein, “about X” (where X is a numerical value) preferably refers to ±10% of the recited value, inclusive. For example, the phrase “about 8” can refer to a value of 7.2 to 8.8, inclusive. This value may include “exactly 8”. Where present, all ranges are inclusive and combinable. For example, when a range of “1 to 5” is recited, the recited range should be construed as optionally including ranges “1 to 4”, “1 to 3”, “1-2”, “1-2 & 4-5”, “1-3 & 5”, and the like. In addition, when a list of alternatives is positively provided, such a listing can also include embodiments where any of the alternatives may be excluded. For example, when a range of “1 to 5” is described, such a description can support situations whereby any of 1, 2, 3, 4, or 5 are excluded; thus, a recitation of “1 to 5” may support “1 and 3-5, but not 2”, or simply “wherein 2 is not included.”

The entire disclosures of each patent, patent application, and publication cited or described in this document are hereby incorporated herein by reference.

Esketamine is the S-enantiomer of the racemic cyclohexanone derivative ketamine, a glutamate N-Methyl-D-aspartate (NMDA) receptor antagonist. It is indicated as an anesthetic, and is approximately twice as potent as an anesthetic as racemic ketamine. The chemical structure of esketamine is shown below:

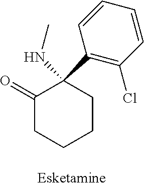 [SMILES: CN[C@]1(c2ccccc2Cl)CCCCC1=O]

Esketamine is a schedule III controlled substance, and can produce dissociative and hallucinogenic effects when subjected to misuse or abuse. Thus, there exists a risk of abuse with respect to dosage forms containing esketamine.

The present disclosure relates, inter alia, to oral dosage forms comprising: (i) a first population of core-shell particles, each of the core-shell particles of the first population comprising a core that includes a gelling polymer and a wax; an active pharmaceutical layer surrounding the core, the active pharmaceutical layer comprising esketamine; and at least one layer surrounding the active pharmaceutical layer, the at least one layer comprising a pH-sensitive film comprising a pH-sensitive polymer that is insoluble in water at a pH greater than 5; and, (ii) a matrix comprising a carbomer gelling polymer and sodium bicarbonate, wherein the carbomer gelling polymer and sodium bicarbonate are present in said dosage form in a ratio by weight percentage of about 2:2 based on the total weight of the dosage form. The disclosed dosage forms can exhibit an immediate release profile of the esketamine when administered to a human in therapeutic doses, and an extended release profile of the esketamine when administered to a human in supratherapeutic doses.

Unless otherwise specified, “esketamine” as used throughout the present disclosure can also refer to isotopically-enriched esketamine. “Esketamine” as used herein may also refer to that compound in its free base form, or to a salt of the compound, such as esketamine hydrochloride.

As used herein, esketamine that is “isotopically-enriched” refers to a condition in which the abundance of deuterium (²H) ¹³C, or ¹⁵N at any relevant site of the compound is substantially more than the abundance of deuterium, ¹³C, or ¹⁵N naturally occurring at that site in an amount of the compound. A relevant site in a compound as used above is a site which would be designated as “H” or “C” or “N” in a chemical structure representation of the compound when not enriched. The expression, “naturally occurring,” as used above refers to the abundance of the particular atom which would be present at a relevant site in a compound if the compound was prepared without any affirmative synthesis step to enrich the abundance of a different isotope. Thus, for example in a “deuterium-enriched” compound, the abundance of deuterium at any relevant site in the chemical structure of the esketamine can range from an amount that is substantially more than the natural abundance of deuterium (about 0.0115%) all the way up to 100%, for example, from about 1% to about 100%, or from about 10% to about 100%, or from about 50% to about 100%, or from about 90% to about 100%.

Similarly, for a “¹³C-enriched” compound, the abundance of ¹³C at any relevant site in the chemical structure of the API can range from an amount that is substantially more than the natural abundance of ¹³C (about 1.109%) all the way up to 100%, for example, from about 5% to about 100%, or from about 10% to about 100%, or from about 50% to about 100%, or from about 90% to about 100%. Similarly for a “¹⁵N-enriched” compound, the abundance of ¹⁵N at any relevant site in the chemical structure of the esketamine can range from an amount that is substantially more than the natural abundance of ¹⁵N (about 0.364%) all the way up to 100%, for example, from about 1% to about 100%, or from about 10% to about 100%, or from about 50% to about 100%, or from about 90% to about 100%.

Isotopically-enriched compounds can generally be prepared by conventional techniques known to those skilled in the art. Such isotopically-enriched compounds can also be prepared by adapting conventional processes as described in the scientific literature for synthesis of esketamine as suitable for formulation according to the invention, and using an appropriate isotopically-substituted reagent (or reagents) in place of the corresponding non isotopically-substituted reagent(s) employed in the conventional synthesis of the non isotopically-enriched compounds. Examples of ways to obtain a deuterium-enriched compound include exchanging hydrogen with deuterium or synthesizing the compound with deuterium-enriched starting materials.

As used herein, expressions such as “abuse deterrent” and “abuse resistant”, and “preventing”, “deterring”, “resisting”, or “inhibiting” abuse, relate to the ability of features of the claimed formulations to provide significant physical or chemical impediments to the use of an active pharmaceutical ingredient for objectives other than its primary therapeutic indications. The objective in such deterrence includes both making abuse practices significantly more difficult to carry out, and making any product resulting from an attempt to carry out such abuse practices on the claimed formulations significantly less desirable, less profitable, and less abusable to the potential abuser.

The term “immediate release”, as used herein, refers to a dosage form that upon oral ingestion by a human releases substantially all of a contained active pharmaceutical ingredient into a gastrointestinal tract for biological uptake in a short time. In vitro methods of measuring a release profile of a dosage form, for the purpose of determining whether a dosage form exhibits an immediate release or extended release dissolution profile, are known in the pharmaceutical arts. By such methods, examples of immediate release dosage forms as described herein can be measured to be capable of releasing substantially all of a total amount of at least one type of active pharmaceutical ingredient (e.g., esketamine) contained in the dosage form (e.g., at least 75, 80, 90, 95, 97 or 100 weight percent of the total amount of the API in a dosage form) into a solution (e.g., acidic aqueous solution) of a suitable pH within 240 minutes, e.g., in less than 180 minutes, less than 90 minutes, or less than 60, 30, 15, or 5 minutes. For example, a release profile of a dosage form of the present description may be measured by a method that exposes the dosage form to a volume of up to 900 mL (e.g., 300 mL, 500 mL, or 900 mL, based on various test methods) of hydrochloric acid (from 0.01 to 0.1N), e.g., aqueous hydrochloric acid, at a pH of from 1 to 2, and at a temperature of 37 degrees Celsius. According to some embodiments, the dosage forms described herein release at least 90% or at least 95% of API in less than 30 minutes when administered at therapeutic doses (e.g., as a single dosage unit), wherein the release profiles may be evaluated, for example, by dissolution in 500 mL of 0.01N HCl media using USP II apparatus at 50 RPM paddle speed and 37° C. In some embodiments, the 0.01N HCl medium is subjected to deaeration based on the recommendations of USP <1092>. A release profile of a dosage form of the present description may alternatively be measured by a method that exposes the dosage form to a volume of up to 900 mL (e.g., 200 mL, 300 mL, 400 mL, 500 mL, 600 mL, 700 mL, 800 mL, or 900 mL, based on various test methods) of an aqueous buffer solution (e.g., an acetate buffer solution) at a pH that is representative of the pH conditions of a fed stomach, e.g., at a pH of about 4.5, and at a temperature of 37 degrees Celsius, with or without deaeration.

The term “extended release” can be defined as not more than 75% release of the API at 30 minutes, wherein the release profiles may be evaluated, for example, by dissolution in 400 mL of 0.1N HCl media using USP II apparatus at 50 RPM paddle speed and 37° C. In some embodiments, the 0.1N HCl medium is subjected to deaeration based on the recommendations of USP <1092>.

According to some embodiments, the dosage forms described herein demonstrate (i) not less than 95% of API released in 30 minutes when administered at therapeutic doses, wherein the release profile is evaluated by dissolution in 500 mL of deaerated 0.01N HCl media using USP II apparatus at 50 RPM paddle speed and 37° C.; and (ii) not more than 75% release of the API at 30 minutes when administered at supratherapeutic doses, wherein the release profiles may be evaluated by dissolution in 400 mL of deaerated 0.1N HCl media using USP II apparatus at 50 RPM paddle speed and 37° C. In this context, a “supratherapeutic dose” can be understood to correspond to administration of five or more, six or more, seven or more, eight or more, nine or more, ten or more, eleven or more, or twelve or more individual dose units, e.g., tablets, simultaneously. It will also be understood that administering multiple individual dose units simultaneously would reasonably include administering those multiple doses sequentially over a short time interval, e.g., over an interval of less than 60 minutes, less than 30 minutes, less than 15 minutes, less than 5 minutes, or less than one minute.

Dosage forms as described herein can be formulated to provide an immediate release profile of esketamine, while including effective or advantageous abuse deterrent features that are effective to deter abuse of the API. The combination of immediate release of esketamine with broad abuse resistance of the same API for multiple abuse modalities including multi-tablet dosing, as described herein, is not believed to be previously known. The present dosage forms can also be more specifically characterized as resistant to certain common methods of abuse, such as 1) abuse by injection (e.g., by steps that include grinding a dosage form and dissolving it), 2) abuse by nasal insufflation (e.g., also by grinding and optionally dissolving the dosage form), and 3) abuse by multi-tablet dosing by oral consumption, meaning simultaneous oral ingestion of multiple, supratherapeutic quantities of orally administered dosage forms such as tablets or capsules. The third mode of abuse, multi-tablet dosing, is particularly common with immediate release dosage forms and is particularly difficult to defend against by design of a dosage form structure or by formulation. Accordingly, the ability of the presently-described dosage forms to prevent or deter abuse (or even accidental overdose) by multi-tablet dosing represents a particularly noteworthy feature.

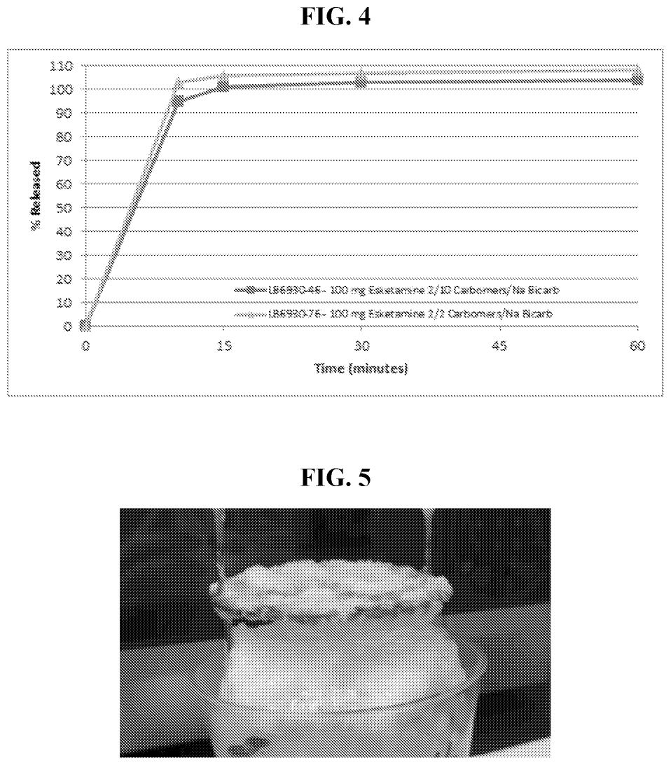

In vitro testing as described herein demonstrated that the presently disclosed dosage forms provide deterrence against abuse by multi-tablet dosing. More specifically, in vitro testing of exemplary dosage forms was performed by conducting dissolution testing of one or more dosage forms (tablets) in 400 mL of deaerated 0.1N HCL maintained at 37 degrees Celsius using a 50 RPM paddle speed. See Example 5, infra. As shown in FIG. 4 and FIG. 5, the amount (percentage per tablet) of esketamine released in the media is reduced when multiple dosage units are administered together, as opposed to when a single dosage unit is subjected to release testing. The data also demonstrate that the tested dosage forms are effective to prevent increased levels of esketamine uptake by an individual who would accidentally ingest multiple tablets, and are thereby effective to prevent or reduce the risk of an unintentional overdose of the esketamine.

A “supratherapeutic dose” refers to a dose that exceeds what would normally be prescribed for therapy with respect to a particular disease or disorder, e.g., a dose that results in a Cₘₐₓ that exceeds what would normally be needed for effective therapy of the particular disease or disorder. For example, a supratherapeutic dose may represent four, five, six, seven, eight, nine, ten, eleven, twelve, or more than twelve individual dosage units (e.g., tablets, capsules, and the like).

As used herein, the phrase “administered in a manner intended to result in administration of the esketamine in a higher than therapeutic dose” includes administration in a manner that would result in a Cₘₐₓ of esketamine that is higher or significantly higher than a therapeutic Cₘₐₓ of esketamine considered to be safe and efficacious for treating a particular disease or disorder, if the esketamine was administered in a dosage form that did not include the features of a dosage form described herein.

Dosage forms as described herein can include one or more gelling polymers. A gelling polymer can act as an abuse deterrent feature by compromising abuse practices involving dissolution of the active pharmaceutical ingredient of the dosage in a small volume of solvent in an attempt to render the API more accessible or easily isolatable. A gelling polymer can deter or prevent abuse of the esketamine by increasing the viscosity of a combination of the ground dosage form with solvent to an extent that is sufficient to prevent the combination or the esketamine from being taken up by and injected using a syringe. When exposed to a volume of solvent such as a C₁₋₄ alcohol (e.g., ethanol) or water, a gelling polymer from a ground dosage form can form a non-injectable mass that ranges in type from an insoluble mass, to a gel, to a viscous slurry, that exhibits a viscosity that substantially prevents uptake by or injection from a needle of a hypodermic syringe.

Suitable gelling polymers include one or a combination of polymers that, as part of a dosage form, upon contact of the dosage form with a volume of solvent, absorb the solvent and swells to form a viscous or semi-viscous substance that significantly reduces or minimizes the amount of free solvent that can contain an amount of a solubilized esketamine and that can be drawn into a syringe. The gelled polymer can also or alternatively function to reduce the overall amount of drug that is extractable with the solvent by entrapping the drug in a gel matrix.

At the same time, the gelling polymers used herein do not interfere with desired dissolution of the dosage forms, the desired release (immediate release) of esketamine from the dosage forms, or the uptake of the esketamine by a patient ingesting the intact immediate release dosage form for an intended therapeutic purpose. The gelling polymer may be present in the dosage forms within a core-shell particle that includes active pharmaceutical ingredient, such as in a core or in a layer (“shell”) surrounding the core, wherein an amount of active pharmaceutical ingredient is contained within the core, in a layer that is coated core, or both. Another exemplary location is within a matrix. Gelling polymer may also or alternatively be present in a core-shell particle that does not include esketamine, such as in the core, or in a layer surrounding the core.

The gelling polymer can be present in a dosage form at any desired amount and within any portion of the present dosage forms. The amount of gelling polymer can be any useful amount, meaning an amount that can produce an abuse-deterrent viscous mixture or gel if the dosage form is crushed, ground, powdered, or otherwise similarly manipulated, and mixed with solvent. A useful amount of total gelling polymer in a dosage form may be in a range from 0.5 to 90 weight percent gelling polymer based on a total weight of the dosage form, e.g., from 0.7 to 20, 1 to 20, 2 to 15, 2 to 10, or 3 to 7 weight percent gelling polymer based on total weight of the dosage form.

As used throughout the present disclosure, “crushing” or “crushed” may refer to a sample that has been physically manipulated by mechanical means thereby leading to the sample's partial or complete disintegration, wherein the manipulation takes place for a time period of up to about 90 seconds and wherein at the completion of the manipulation, less than 10% of the recovered sample has a particle size of >500 μm and, by weight, more than about 99% of the sample can be recovered.

The presently disclosed dosage forms include core-shell particles, and the cores of such particles preferably contain a gelling polymer. A core (uncoated) of a core-shell particle can contain any useful amount of gelling polymer, up to and including 100 percent gelling polymer in a core of a core-shell particle, e.g., from 10 to 95 weight percent gelling polymer based on the total weight of the core, such as 20 to 90, 25 to 85, 30 to 85, 40 to 85, 40 to 80, 45 to 75, 50 to 75, 55 to 70, 55 to 65, or 57 to 62 weight percent gelling polymer based on the total weight of the core.

Described in terms of total weight of a dosage form, an amount of gelling polymer present in a core of a core shell polymer may be, e.g., in a range from 0.5 to 15 weight percent gelling polymer (present in the core) per total weight of the dosage form, such as from 1 to 10 weight percent gelling polymer (present in the core) per total weight dosage form.

In certain embodiments, gelling polymer may also or alternatively be provided in the matrix portion of the present dosage forms. The types of gelling polymers in the respective portions of the present dosage forms may be the same or different. For example, when present, the gelling polymer in the core of the core-shell particles may be the same or a different type as a gelling polymer in the matrix of the dosage form.

A gelling polymer for use in the present dosage forms can be any polymeric material that exhibits the ability to retain a significant fraction of adsorbed solvent in its molecular structure, e.g., the solvent being a solvent otherwise useful by an abuser to extract API from a dosage form or a crushed or powdered dosage form, the solvent for example being water or a C₁ to C₄ alcohol such as ethanol or methanol. Exemplary gelling polymers include materials that can swell or expand to a very high degree when placed in contact with such a solvent. The swelling or expansion may cause the gelling polymer to experience from a two- to one-thousand-fold volume increase relative to the dry state. More specific examples of gelling polymers include swellable polymers sometimes referred to as osmopolymers or hydrogels. The gelling polymer may be non-cross-linked, lightly crosslinked, or highly crosslinked. The crosslinking may involve covalent or ionic bonds with the polymer possessing the ability to swell in the presence of a solvent, and when cross-linked will not dissolve in the solvent.

A gelling polymer, upon dissolution or dispersion in an aqueous solution or dispersion (e.g., water) at a concentration of 2% w/w (based on the dry material), preferably creates a solution/dispersion with a viscosity of from about 100 to about 200,000 mPa·s (e.g., 4,000 to 175,000 mPa·s, and 4,000 to 50,000 mPa·s) as measured at 20 degrees Celsius (+/−0.2 degree Celsius) using the analysis method described in the USP 33 monograph for Hypromellose, incorporated herein by reference.

Generally suitable gelling polymers include pharmaceutically acceptable polymers that undergo an increase in viscosity upon contact with a solvent, as described. Various examples of polymers are known to be useful in this manner, generally including natural and synthetic starches (i.e., modified or pregelatinized modified starch), natural and synthetic celluloses, acrylates, and polyalkylene oxides. Examples of natural starches include natural starches include corn starch, potato starch, rice starch, tapioca starch and wheat starch, hydroxypropyl starch such as hydroxypropyl corn starch, hydroxypropyl pea starch and hydropropyl potato starch (derivative of natural starch). Examples of synthetic starches, i.e., modified or pregelatinized modified starch, include acetylated distarch adipate, waxy maize basis, acid-treated maize starch, acid-treated waxy maize starch, distarch phosphate, waxy maize basis, oxidized waxy maize starch, and sodium octenyl succinate starch. Examples of celluloses include carboxymethyl cellulose calcium, carboxymethylcellulose sodium, ethycellulose, methylcellulose, cellulose ethers such as hydroxypropyl cellulose, hydroxyethylcellulose, hydroxy ethyl methyl cellulose, hydroxypropyl methyl cellulose, carboxymethylcellulose sodium, and low substituted hydroxypropyl cellulose. Examples of acrylates include Eudragit RS, RL, NE, NM. Examples of polyalkylene oxides include polyethylene oxide such as POLYOX N10, N80, N60K, WSR-1105 LEO, or WSR-301 LEO, or WSR-303 LEO.

Accordingly, examples of suitable gelling polymers for use in any component of the present dosage forms include, among others, polyethylene oxide, polyvinyl alcohol, hydroxypropyl methyl cellulose, hydroxypropyl cellulose, methyl cellulose, hydroxyethyl methyl cellulose, sodium carboxymethylcellulose, hydroxyethyl cellulose, and polyacrylic acid, and other high molecular weight polymers capable of attaining a viscosity level effective to prevent uptake in a syringe, if combined with a small volume of solvent as described.

Other examples of suitable gelling polymers can include, if of sufficiently high molecular weight: ethylcellulose, cellulose acetate, cellulose acetate propionate, cellulose acetate butyrate, cellulose acetate phthalate and cellulose triacetate, cellulose ether, cellulose ester, cellulose ester ether, cellulose; acrylic resins comprising copolymers synthesized from acrylic and methacrylic acid esters, for example acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate copolymers.

Exemplary gelling polymers also include natural polymers such as those derived from a plant or animal, as well as polymers prepared synthetically. Examples include polyhydroalkylcellulose having a molecular weight greater than 50,000; poly(hydroxyalkylmethacrylate) having a molecular weight of from 5,000 to 5,000,000; poly(vinyl-pyrrolidone) having a molecular weight of from 100,000 to 3,000,000; anionic and cationic hydrogels; poly(electrolyte) complexes; poly(vinyl alcohol) having a low acetate residual; a swellable mixture of agar and carboxymethyl cellulose; a swellable composition comprising methyl cellulose mixed with a sparingly cross-linked agar; a polyether having a molecular weight of from 10,000 to 6,000,000; water-swellable copolymer produced by a dispersion of finely divided copolymer of maleic anhydride with styrene, ethylene, propylene, or isobutylene; water swellable polymer of N-vinyl lactams; and the like.

Other polymers useful as gelling polymers include pectin having a molecular weight ranging from 30,000 to 300,000; polysaccharides such as agar, acacia, karaya, tragacanth, algins and guar; polyacrylamides; water-swellable indene maleic anhydride polymers; Good-rite® polyacrylic acid having a molecular weight of 80,000 to 200,000; Polyox® polyethylene oxide polymers having a molecular weight of 100,000 to 7,000,000; starch graft copolymers; Aqua-Keep® acrylate polymers with water absorbability of 400 times its original weight; diesters of polyglucan; a mixture of cross-linked polyvinyl alcohol and poly(-vinyl-2-pyrrolidone); poly(ethylene glycol) having a molecular weight of 4,000 to 100,000.

In certain embodiments, a gelling polymer may include hydroxypropyl methyl cellulose (e.g., Hypromellose or HPMC), and hydroxy methyl cellulose, methyl cellulose, hydroxyethylmethyl cellulose, or sodium carboxymethyl cellulose. When present, a hydroxypropyl methyl cellulose can have a molecular weight ranging from 10,000 to 1,500,000. Examples of suitable, commercially available hydroxypropyl methylcellulose polymers include HPMC K100M, Methocel K100LV and Methocel K4M.

A specific class of gelling polymer of which one or more members may be used in the present dosage forms is carbomer polymers, which are polymers derived from acrylic acid (e.g., acrylic acid homopolymers) and crosslinked with polyalcohol allyl ethers, e.g., crosslinked with polyalkenyl ethers of pentaerythritol or sucrose. Carbomer polymers are hydrophilic and are not substantially soluble in water. Rather, these polymers swell when dispersed in water forming a colloidal, mucilage-like dispersion. Carboxyl groups provided by acrylic acid residues of the polymer backbone are responsible for certain behavior of the polymers. Particles of this polymer can be viewed as a network structure of polymer chains interconnected by crosslinks. The structure can swell in water by up to one thousand times of an original (dry) volume (and ten times an original diameter of polymer particles) to form a gel when exposed to a pH environment above 4-6. The pKa of these polymers can be 6±0.5. Accordingly, carboxylate groups pendant from the polymer backbone can ionize at a pH above 6, producing a repulsion between the negatively-charged particles, which adds to the swelling of the polymer if exposed to solvent at this pH range.

For this reason, the presently disclosed dosage forms can include a pH adjuster in an amount and location within the dosage form to raise the pH of a carbomer polymer to at least 6, to substantially neutralize the carboxylate groups. Exemplary types and amounts of pH adjusters are discussed more fully, infra.

Carbomer polymers are often referred to in the art using alternative terminology such as, for example, carbomer homopolymer, acrylic acid polymers, carbomer, carboxy polymethylene, carboxyvinyl polymer, polyacrylic acid, and poly(acrylic acid), The USP-NF lists three umbrella monographs i.e. for “carbomer copolymer,” for “carbomer homopolymer,” and for “carbomer interpolymer.”

Certain carbomer polymers that may be useful as a gelling polymer can have an average equivalent weight of 76 per carboxyl group. Examples of suitable commercially available carbomers include Carbopol® 934, 934P NF, Carbopol® 974P NF and Carbopol® 971P NF, Carbopol® 940, and Carbopol® 941, Carbopol® 71G, commercially available from Lubrizol. Examples of such polymers are described in U.S. Pat. Nos. 2,798,053 and 2,909,462, the entireties of which are incorporated herein by reference. Theoretical molecular weight ranges of Carbopol® products are in a range from 700,000 to 3 billion, theoretical estimation. For dosage forms as described herein, a gelling polymer (e.g., Carbopol®) can have a molecular weight and viscosity-increasing performance that will reduce or substantially inhibit an ability of an abuser to extract API from a combination of dosage form and a small volume of solvent, as described, while also being capable of being processed into a compressed dosage form.

A gelling polymer can also be characterized by viscosity of a solution prepared from the gelling polymer. Product information for commercially available Carbopol® polymers reports that viscosities of different Carbopol® polymers are as follows:

| Type of | Viscosity |
|---|---|
| Carbomer | specified (cP) |
| Carbomer Homopolymer Type A | 4,000-11,000 |
| (compendial name for Carbopol ® 71G, | |
| Carbopol ® 971P and Carbopol ® 981) | |
| Carbomer Homopolymer Type B | 25,000-45,000 |
| (compendial name for Carbopol ® 934P, | |
| and Carbopol ® 934) | |
| Carbomer Homopolymer Type C | 40,000-60,000 |
| (compendial name for Carbopol ® 980) | |

(Type A and Type B viscosities measured using a Brookfield RVT, 20 rpm, neutralized to pH 7.3-7.8, 0.5 weight percent mucilage, spindle #5.)

A further exemplary gelling polymer is the class of xanthan gum polymers, which includes natural polymers useful as hydrocolloids, and derived from fermentation of a carbohydrate. A molecular weight of a Xanthan gum may be approximately 1,000,000. Xanthan gum has been shown to provide particularly useful extraction resistance in a dosage form as described, and therefore may be preferred in dosage forms as described, especially if present in an amount of at least 2 or 3 weight percent based on a total weight of a dosage form.

Without limiting the scope of useful gelling polymers to any specific type or molecular weight, examples of useful gelling polymers, and useful respective molecular weights, are shown below.

| Gelling | Weight Average |
|---|---|
| Polymer | Molecular Weight |
| Carbomer | 700,000 to 3 billion (estimated) |
| HPMC 2910 K types | 164,000-1,200,000 |
| HPMC 2910 E types | 20,000-746,000 |
| hydroxyethylcellulose | 90,000-1,300,000 |
| ethylcellulose | 75,000-215,000 |
| carboxymethylcellulose | 49,000-725,000 |
| sodium carboxymethylcellulose | 49,000-725,000 |
| povidone | 4,000-1,300,000 |
| copovidone | 47,000 |
| hydroxypropyl cellulose | 40,000-1,150,000 |
| xanthan gum | 1,000,000 |
| polyethylene oxide | Average molecular wt: |
| | 100,000-7,000,000 |

The present dosage forms may optionally include another abuse deterrent feature in the form of a wax, such as a wax/fat material, e.g., as described in U.S. Pat. No. 8,445,018, the entirety of which is incorporated herein by reference. The wax can be a solid wax material that is present in the dosage form at a location that inhibits an abuser from crushing, grinding, or otherwise forming the dosage form into a ground powder that might be abused by a nasal insufflation mode, or from which active pharmaceutical agent can be easily accessed and removed such as by dissolution or extraction using a solvent.

A wax may be present in the dosage form at a location and in an amount to also not interfere with desired uptake of the active pharmaceutical ingredient by a patient upon oral ingestion, in an immediate release dosage form. An exemplary location is at a core of a core-shell particle, especially a core that also contains gelling polymer and that either may or may not contain active pharmaceutical ingredient. In one embodiment, a wax is provided in the core of core-shell particles in the present dosage forms, along with a gelling polymer, in the absence of esketamine in the core. Wax located at a core of a particle (e.g., a core-shell particle) that also includes active pharmaceutical ingredient (e.g., at a layer covering the core, or within the core) will become mixed with the active pharmaceutical ingredient upon crushing or grinding, etc., of the particle. In one embodiment, a wax is provided in the core of core-shell particles in the present dosage forms, along with a gelling polymer, in the absence of esketamine in the core. Wax that is located at a core of such a particle (e.g., a core-shell particle) wherein the core does not contain API will also become mixed with the API (e.g., API present in API-containing core-shell particles that are also present in the dosage form) upon destructive manipulation (e.g., crushing or grinding) of the dosage form. When the wax is mixed with the active pharmaceutical ingredient, the active ingredient is inhibited or prevented from becoming thereafter dissolved in a solvent such as water, or otherwise efficiently accessed by an abuser.

A core (uncoated) of a core-shell particle can contain any useful amount of wax, up to and including 100 percent wax, e.g., from 0.1 to 85 weight percent wax based on the total weight of the core, such as 5 to 80, 10 to 70, 15 to 60, 20 to 50, 20-40, or 20-30, or in an amount of about 5, 10, 15, 20, 22, 24, 25, 26, 27, 28, 30, 35, 40, 45, 50, 55, 60, 65, 70, 75, or 80 weight percent wax, based on the total weight of the core.

The wax may be a wax (e.g., fat) material that is generally hydrophobic and that may be either solid or liquid at room temperature, preferably solid at room temperature (25 degrees Celsius). Generally useful fats include those hydrophobic materials that are fatty acid-based compounds generally having a hydrophilic/lipophilic balance (HLB) of 6 or less, more preferably 4 or less, and most preferably 2 or less. A fat can have any melting temperature, with preferred fats being solid at room temperature and having a melting point that is at least 30 degrees Celsius, e.g., at least 40 degrees Celsius, e.g., at least 50 degrees Celsius. Useful fats include fatty acids and fatty esters that may be substituted or unsubstituted, saturated or unsaturated, and that have a chain length of at least 10, 12, or 14 carbons. The esters may include a fatty acid group bound to any of an alcohol, glycol, or glycerol. With regard to glycercols, for example, mono-, di-, and tri-fatty substituted glycerols can be useful as well as mixtures thereof.

Suitable wax ingredients include fatty acid esters, glycerol fatty acid esters, fatty glyceride derivatives, waxes, and fatty alcohols such as, for example, glycerol behenate (also referred to as glyceryl behenate, glycerin behenate, or glycerol docosanoate) (available commercially as COMPRITOL®), glycerol palmitostearate (PRECIROL®), glycerol monostearate, stearoyl macroglycerides (GELUCIRE® 50/13). Other waxes more generally include insect and animal waxes, vegetable waxes, mineral waxes, petroleum waxes, and synthetic waxes; particularly examples include beeswax, carnauba wax, candelilla wax, montan wax, ouricury wax, rice-bran wax, jojoba wax, microcrystalline wax, cetyl ester wax, cetyl alcohol, anionic emulsifying wax, nonionic emulsifying wax and paraffin wax.

The dosage form may optionally include another component contributing to abuse deterrence in the form of a filler or binder material provided in a manner to compromise abuse practices wherein an abuser crushes, grinds, or otherwise forms the dosage form into a ground powder that might be abused by nasal insufflation, or from which active pharmaceutical agent can be easily accessed and removed such as by dissolution or extraction using a solvent.

The binder or filler may be present in the dosage form at a location and in an amount to not interfere with desired uptake of the active pharmaceutical ingredient by a patient upon oral ingestion, in an immediate release dosage form. An exemplary location is at a core of a core-shell particle. Suitable filler or binder located at a core of a core-shell particle that also includes active pharmaceutical ingredient (such as in a layer covering the core, or within the core) will become mixed with the active pharmaceutical ingredient upon destructive manipulation (e.g., crushing or grinding) of the particle. As discussed previously, the dosage form may also include core shell particles that do not contain esketamine. A filler or binder that is located at a core of such a particle that does not contain API will also become mixed with the API (e.g., API present in API-containing core shell particles that are also present in the dosage form) upon manipulation (e.g., crushing or grinding) of the dosage form. When a filler or binder is mixed with the active pharmaceutical ingredient, the active pharmaceutical ingredient is inhibited or prevented from becoming thereafter dissolved in a solvent such as water or otherwise efficiently accessed by an abuser.

When present within the core of a core-shell particle of the present dosage forms, filler or binder may be present in any useful amount, up to and including 100 percent filler or binder (singly or in combination) in a core of a core-shell particle. For example, filler or binder may be present in the core of a core-shell particle in an amount of from 5 to 95 weight percent filler or binder based on total weight of the core, such as from 5 to 70, 5 to 50, 7 to 40, 10 to 30, 10 to 20, 10 to 17, 12 to 17, or 13 to 16 weight percent, or in an amount of about 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, or 80 percent by weight based on the total weight of the core. Examples of cores that contain high levels of filler include spherical particles that contain 100 percent sugar, and spherical particles that contain 100 percent microcrystalline cellulose. Inert spherical filler products such as these, having useful particle sizes, are commercially available under the trade name Celphere®, and under the trade name Suglets® (sugar spheres, also containing starch), including as follows: CELPHERE SCP-100 (Particle size (μm) 75-212); CELPHERE SCP-102 (Particle size (μm) 106-212); CELPHERE SCP-203 (Particle size (μm) 150-300); CELPHERE SCP-305 (Particle size (μm) 300-500); CELPHERE SCP-507 (Particle size (μm) 500-710); CELPHERE SCP-708 (Particle size (μm) 710-850). The particle sizes of these can be considered to be useful for any core as described herein, prepared of any single filler, gelling polymer, binder, any combination thereof, or any single or combination of materials combined with API.

A pH-sensitive layer may be useful as a solvent-resistant film, placed in a dosage form as a layer of a core-shell particle to surround, cover, or enclose a portion of the core-shell particle that contains active pharmaceutical ingredient. For example, in a core-shell particle, an active pharmaceutical ingredient may be located at a core or in a layer outside of an uncoated or coated core, and a solvent-resistant film in the form of a pH-sensitive layer may be disposed as a separate layer surrounding or covering the portion of the core-shell particle that contains the active pharmaceutical ingredient. The pH-sensitive layer may be in direct contact with (adjacent to) a core or a layer that includes active pharmaceutical ingredient. Alternatively, a core-shell particle may include one or more intermediate layers between a pH-sensitive layer and a core or layer that includes active pharmaceutical ingredient. In addition, a pH-sensitive layer may be included in the dosage form as a layer of a core-shell particle that does not contain either an API layer or any API.

A useful pH-sensitive layer may include a polymer or other material that can be placed as a layer of a particle as described herein, such as to cover a more inner layer or core that contains active pharmaceutical ingredient, to form a pH-sensitive film surrounding or covering active pharmaceutical ingredient. The pH-sensitive film can be solubilized by exposure to a liquid that exhibits a pH below 7.

Examples of pH-sensitive polymers that may be used in a pH-sensitive layer in the present dosage forms include the class of reverse enteric polymers that contain cationic-functional groups and that exhibit pH-dependent solubility as described herein. Examples include polymers that contain basic functional groups, such as amino groups, and that exhibit solubility at pH conditions found in beverages such as wine, other alcoholic beverages, soft drinks, and the like. More specific examples of such pH-sensitive polymers include copolymers of dimethyl aminoethyl methacrylates, and neutral methacrylic acid esters; e.g., dimethyl aminoethyl methacrylate, butyl methacrylates, and methyl methacrylates, such as at a ratio of 2:1:1. Examples of such polymers are commercially available under the trade name Eudragit® E 100, Eudragit® E PO, Eudragit® E 12.5, and similar amino-functional pH-sensitive polymers. A preferred pH-sensitive polymer is the polymer Eudragit E 100, but any polymer that is sufficiently hydrophilic at a low pH and hydrophobic at a higher pH to exhibit pH-dependent solubility, may also be effective if otherwise acceptable for use in a pharmaceutical dosage form, for example, as a non-toxic ingredient of an oral dosage form. Reverse enteric compositions are also described in EP 1694724 B 1, titled “pH Sensitive Polymer and Process for Preparation Thereof”, incorporated herein by reference.

When present in a coating layer of a core-shell particle, whether that particle contains active pharmaceutical ingredient or not, a solvent-resistant film layer may be present at any amount useful as an abuse deterrent feature, such as in a range from 0.1 to 90 weight percent of a total weight of a core-shell particle, e.g., from 3 to 50, 10 to 50, 20 to 50, 25 to 45, 25 to 40, 30 to 40, or 30 to 35 weight percent, or in an amount of about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 weight percent, relative to the total weight of the core-shell particle. More generally, a useful amount solvent-resistant film layer or polymer in a dosage form may be in a range from 1 to 50 weight percent solvent-resistant film layer or polymer based on a total weight of a dosage form, e.g., from 2 to 30 or from 3 to 15 weight percent solvent-resistant polymer based on total weight dosage form.

A dosage form as presently described further includes a disintegrant, which functions to cause the dosage form to expand and break up during use, e.g., under the conditions within a human stomach, to allow the active pharmaceutical ingredient of the dosage form to be released in a manner to achieve an immediate release profile. Disintegrants are known ingredients of pharmaceutical dosage forms, with various examples being known and commercially available. Examples of disintegrants include compositions of or containing sodium starch glycolate, starch (e.g., maize starch, potato starch, rice starch, tapioca starch, wheat starch, corn starch and pregelatinized starch), croscarmellose sodium, crospovidone (crosslinked polyvinyl N-pyrrolidone or PVP) (polyplasdone XL-10), sodium starch glycolate (EXPLOTAB® or PRIMOJEL®), any combination of the foregoing, and other pharmaceutically acceptable materials formed into particles having a particle size, density, and other characteristics that are suitable to allow processing of the disintegrant into a useful immediate release dosage form.

The disintegrant can be present in an immediate release dosage form at any location that allows the disintegrant to function as desired, to expand within the intact dosage form, upon ingestion, to cause the ingested dosage form to break apart and allow for desired immediate release of active pharmaceutical ingredient from the dosage form, in a stomach. An exemplary location for the disintegrant can be within the matrix, where it can function as an excipient used to contain the core-shell particles in a dosage form such as a compressed tablet or capsule.

When included as an excipient of a dosage form, a disintegrant may be present in an amount useful to achieve immediate release of an API of a dosage form. For example, the disintegrant may be present in an amount of 0.5 to 50, 5 to 40, 10 to 35, 15 to 30, 15 to 25, 17 to 22, or 19 to 21 weight percent, or in an amount of about 5, 10, 12, 14, 16, 18, 19, 20, 21, 22, 24, 26, 28, 30, 35, 40, 45, or 50 weight percent, based on a total weight of the dosage form. The amount of disintegrant in the matrix of a dosage form can be consistent with these amounts. For example, disintegrant can be included in a matrix (e.g., total of a dosage form that is other than the core-shell particles) of a dosage form in an amount in a range from 0.5 to 50 weight percent disintegrant based on a total weight of the matrix, for example, 1 to 30 weight percent disintegrant based on total weight matrix.

The presently disclosed dosage forms also include a pH adjuster. A pH-adjuster can be included at a location of the dosage form to affect pH at a specific location of the dosage form that is only a portion of a total dosage form. As an example, a pH-adjuster in the form of a base may be included at a location of a gelling polymer that contains acid functionalities, to neutralize the acid functionalities. Suitable agents that can act as a pH-adjuster include, for example, phosphate buffering agents such as, disodium hydrogen phosphate, sodium dihydrogen phosphate and the equivalent potassium salts; carbonate or bicarbonate salts, such as sodium bicarbonate, sodium carbonate, potassium bicarbonate, potassium carbonate, magnesium carbonate and calcium carbonate; hydroxide bases such as, sodium hydroxide, potassium hydroxide, ammonium hydroxide; and amine bases such as, triethanolamine, tromethamine, aminomethyl propanol, and tetrahydroxypropyl ethylenediamine.

The amount of pH-adjuster included at the location of the gelling polymer can be an amount effective to neutralize the acid functionalities of the gelling polymer at that location. More specifically, a component of a dosage form as described that includes an acid-functional gelling polymer such as a carbomer may include a base in an amount and location to neutralize the acid functionalities of that polymer. The pH-adjuster can be located at a location effective to cause such neutralization, e.g., at the location of the dosage form that contains the acid-functional gelling polymer, for example at a core of a core-shell particle or as part of a matrix.

According to some embodiments, the pH adjuster is in the present dosage forms in an amount that is from about 0.5 to about 5 percent by weight, from about 1 to about 4 percent by weight, from about 1.5 to about 4 percent by weight, from about 2 to about 3 percent by weight, or in an amount of about 0.5, 1, 1.5, 2, 2.5, 3, 3.5, 4, 4.5, or 5 percent by weight, based on the total weight of the dosage form.

In certain embodiments, the dosage forms are such that when a dual screen apparatus with a top screen and a bottom screen is used to extract at least a portion of a gel that is formed in an aqueous medium comprising 0.025 N HCl and 70 mM NaCl at 37° C. from a supratherapeutic dose of the dosage forms from the medium, a first quantity of the gel adheres to a lower surface of the top screen of the apparatus, and a second quantity of the gel adheres to a upper surface of the bottom screen of the apparatus, and the vertical thickness of the second quantity is at least twice the vertical thickness of the first quantity.

The gels that are formed in an aqueous medium comprising 0.025 N HCl and 70 mM NaCl at 37° C. from a supratherapeutic dose of the present dosage forms may also be characterized as being substantially uniformly dispersed with the medium. This means, for example, that the gel that is formed within the medium does not include a greater concentration of gel within any particular portion of the medium, such as within the upper portion or within the lower portion of the medium. In other words, the concentration of gel within the medium is substantially homogenous upon visual inspection within all portions of the medium, including within the upper portion of the medium and within the lower portion of the medium, respectively. A gel that is substantially uniformly dispersed within the medium in which it is formed may also be characterized by an absence of portions of gel that are interspersed with portions of medium that do not contain gel. This can be described as the condition wherein visually readily apparent clumps of gel are suspended in the medium. As described below, FIG. 8 illustrates the characteristic of a gel formed from dosage forms as disclosed herein of being substantially uniformly dispersed.

The above-described characteristics are indicative of the fact that the gels that are formed from the present dosage forms in a medium as described herein are thick and viscous, with a uniform dispersion within the medium. These attributes, in turn, enhance the ability of the present dosage forms to resist uptake by or injection from a needle of a hypodermic syringe when combined with a solvent in supratherapeutic doses, and also or alternatively to reduce the overall amount of drug that is extractable with a solvent by entrapping the drug in a gel matrix.

A dosage form as described can also include any of various known and conventional pharmaceutical excipients that may be useful to achieve desired processing and performance properties of an immediate release dosage form. These excipients may include disintegrants, fillers, binders, lubricants, glidants, and coloring agents, and can be included in core-shell particles or in a matrix (e.g., compressed matrix) of a tablet or capsule. A more detailed description of pharmaceutical excipients that may also be included in the tablets of the present invention can be found in The Handbook of Pharmaceutical Excipients, 5th ed. (2006). As noted above, one or more of these excipients, such as the binder, filler, or both, may contribute to abuse deterrence in a manner to compromise abuse practices wherein an abuser crushes, grinds, or otherwise forms the dosage form into a ground powder that might be abused by nasal insufflation, or from which active pharmaceutical agent can be easily accessed and removed such as by dissolution or extraction using a solvent.

Examples of fillers that may be useful in an immediate release dosage form as described include lactose, starch, dextrose, sucrose, fructose, maltose, mannitol, sorbitol, kaolin, microcrystalline cellulose, powdered cellulose, calcium sulfate, calcium phosphate, dicalcium phosphate, lactitol or any combination of the foregoing. As compared to non-filler ingredients such as gelling polymers, a filler will have a molecular weight that does not result in a substantial viscosity increase or formation of a gel as described herein for a gelling polymer, if combined with a solvent such as water.

A filler may be present in any portion of a dosage form as described, including a core-shell particle; the filler may be present in a core, in a layer containing an active pharmaceutical ingredient that is disposed on the core, in a solvent resistant film, in the matrix, or in two or more of these portions of the dosage form. The filler may be present at any one or more of these portions of a dosage form in an amount to provide desired processing or functional properties of a portion of the dosage form and of the entire dosage form. The amount of total filler in a dosage form can also be as desired to provide desired functionality, including an immediate release profile, for example, in an amount of greater than 0 to 80, 5 to 50, 5 to 40, 10 to 30, 12 to 25, or 12 to 15 weight percent, or in an amount of about 2, 4, 5, 7, 9, 10, 12, 13, 14, 15, 16, 17, 18, 19, 20, 25, 30, 35, or 40 weight percent filler based upon the total weight of the dosage form.

Examples of binders that may be included in a dosage form as described herein include polymeric material such as alginic acid, sodium carboxymethylcellulose, microcrystalline cellulose, dextrin, ethylcellulose, gelatin, starch, pregelatinized starch, polyvinyl alcohol, polyethylene oxide, polyvinylpyrrolidone, polyacrylamides, polyvinyloxoazolidone, polyvinylalcohols, methylcellulose, hydroxypropyl cellulose, hydroxymethyl cellulose and any combination of two or more of these. A binder may be a water-soluble material. As compared to non-binder ingredients such as a gelling polymer, a binder is of a molecular weight that does not result in formation of a gel or a highly viscous composition upon combining with a small volume of water. A binder can exhibit a relatively low molecular weight as compared to a gelling polymer, and a relatively lower viscosity (e.g., when measured in a 2% aqueous solution). Polymer useful as a binder may typically have a molecular weight of less than 50,000, e.g., less than 30,000, or less than 10,000.

A binder may be present in any portion of the present dosage forms, including in a core or a film or coating of a core-shell particle, or as part of an excipient mixture to contain or bind core-shells particles in a dosage form, i.e., within the matrix of a dosage form as described herein. Filler may be included in a core of a core-shell particle in combination with active pharmaceutical ingredient, gelling polymer or both; as part of an active pharmaceutical layer located over a core or another layer of a core-shell particle; as part of a solvent-resistant film; or, within an excipient mixture (matrix) useful to bind particles into a dosage form. A binder may be present at any one or more of these portions of an immediate release dosage form as described, in an amount to provide desired processing or functional properties in each portion of the dosage form and of the overall dosage form. The amount of total binder in a dosage form can also be as desired to provide desired functionality, including immediate release functionality. For example, a binder may be provided in an amount of about 0.1 to 40, 5 to 40, 10 to 30, 12 to 25, or 12 to 15 weight percent, or in an amount of about 2, 4, 5, 7, 9, 10, 12, 13, 14, 15, 16, 17, 18, 19, 20, 25, 30, 35, or 40 weight percent binder based upon the total weight of the dosage form.

Examples of lubricants include inorganic materials such as talc (a hydrated magnesium silicate; polymers, such as, PEG 4000; fatty acids, such as stearic acid; fatty acid esters, such as glyceride esters (e.g., glyceryl monostearate, glyceryl tribehenate, and glyceryl dibehenate); sugar esters (e.g., sorbitan monostearate and sucrose monopalmitate); glyceryl dibehenate (Compritol® 888 ATO); and metal salts of fatty acids (e.g., magnesium stearate, calcium stearate, and zinc stearate). Accordingly, commonly used lubricants include talc, glyceryl monostearates, calcium stearate, magnesium stearate, stearic acid, glyceryl behenate, polyethylene glycol, poloxamer and combinations of the foregoing. A lubricant may be included in an immediate release dosage form as described, in any useful amount, such as an amount of about 0.1 to 10 weight percent lubricant based on a total weight of a dosage form, e.g., from 0.2 to 7, 0.3 to 5, 0.5 to 3, 0.7 to 2, or 0.9 to 1.5 weight percent, or in an amount of about 0.1, 0.2, 0.3, 0.4, 0.5, 0.6, 0.7, 0.8, 0.9, 1, 2, 3, 4, or 5 weight percent, based on the total weight of the dosage form.

Examples of glidants include colloidal silicon dioxide, untreated fumed silica (e.g., as available under the trade name Cab-O-Sil®), and crystalline or fused quartz. Glidant may be included in an immediate release dosage form as described, in any useful amount. For example, a glidant may be included in an amount of 0.05 to 5, 0.08 to 3, 0.1 to 2, 0.15 to 1.5, 0.15 to 1, or 0.2 to 0.5 weight percent, or in an amount of about 0.01, 0.03, 0.05, 0.1, 0.13, 0.15, 0.17, 0.2, 0.22, 0.25, 0.27, 0.3, 0.35, 0.4, 0.5, 0.7, 0.9, 1.0, 1.2, 1.4, 1.5, 1.6, 1.8, or 2 percent by weight, based on the total weight of the dosage form.

Examples of coloring agents include FD&C-type dyes and lakes, fruit and vegetable extracts, titanium dioxide, iron oxides and mixtures thereof. A coloring agent may be incorporated into a dosage form by blending (e.g., co-milling and blending) the coloring agent with any other ingredient. Alternately, coloring agent may be applied to an outer surface of a dosage form.

Esketamine, alone or in combination with one or more other active pharmaceutical ingredients, are included in the immediate release dosage forms as described herein. The esketamine may be present in its free base form or as a salt. An exemplary esketamine salt is esketamine hydrochloride.

With abuse deterrent features as described herein, some being operative based on specific structural or compositional features of a core-shell particle, the esketamine can be located in the dosage form at a location to cause the API to be subject to abuse deterrent features of the core-shell particles, e.g., at a core or inner layer of a core-shell particle.

The amount of esketamine in the dosage forms disclosed herein can be any useful amount, as is known and as may be found in relevant literature. For example, typical therapeutic amounts of esketamine are 5 mg, 10 mg, 20 mg, 30 mg, 40 mg. 50 mg, 60 mg, 70 mg. 80 mg, 90 mg, or 100 mg. Often, when processed into a suitable immediate release dosage form, the esketamine can be present in such dosage form in an amount normally prescribed, typically 0.5 to 25 percent on a dry weight basis, based on the total weight of the dosage form. In other embodiments, a dosage form contains any appropriate amount of esketamine to provide a therapeutic effect. When present in the core-shell particles of the dosage forms, the amount of esketamine in a core-shell particle may be about 10 to about 40% by weight, based on the total weight of the core-shell particle. For example, the esketamine may be present in an amount of about 10 to 35, 12 to 35, or 15 to 35 weight percent, or in an amount of about 10, 11, 12, 13, 14, 15, 16, 17, 18, 19, 20, 21, 22, 23, 24, 25, 26, 27, 28, 29, 30, 31, 32, 33, 34, or 35 weight percent, based on the total weight of the core-shell particle.

The presently disclosed dosage forms can optionally include one or more additional active pharmaceutical ingredients of a type that is not commonly susceptible to abuse. These additional APIs may be any suitable or desired API, such as those in the class of non-steroidal analgesic drugs. The expression “non-steroidal analgesic drugs” as used herein refers to drugs that include those commonly referred to as non-steroidal anti-inflammatory drugs, or “NSAIDS,” and acetaminophen, which is non-steroidal, but does not act via an inflammation mechanism. Accordingly, the term “non-steroidal analgesic drugs” would include acetaminophen, and also include NSAIDS such as aspirin, ibuprofen, and naproxen. The present dosage forms also exhibit immediate release properties with respect to these APIs that are not commonly subject to abuse. Such APIs can be present in the dosage form at any useful level, typically 0.5 to 25, e.g., 1 to 10 weight percent of the API on a dry weight basis, based on a total weight of the dosage form, e.g., at a level of or between 5, 25, 50, 75, 100, 125, 150, 175, 200, 300, 325, 500, 750 or up to or exceeding 1000 milligram (mg) per dosage form unit. As a general matter, the present dosage forms can contain an appropriate amount of an API that is not commonly subject to abuse in order to provide a therapeutic effect that is associated with such additional API.

The present dosage forms can include one or more of the described abuse deterrent features alone or in combination; e.g., one or more of gelling polymer as part of a core-shell particle (e.g., at a core of the core-shell particle); wax as part of a core-shell particle (e.g., at a core of the core-shell particle); binder or filler as part of a core-shell particle (e.g., at a core of the core-shell particle); a film layer that may optionally be a solvent-resistant film (e.g., pH-sensitive film) as part of a core-shell layer; or gelling polymer as a component of an excipient or binder (i.e., a matrix) used to hold core-shell particles together. With these abuse deterrent features, other types of known abuse deterrent features may not be necessary and may be specifically excluded from the present dosage forms. Thus, certain embodiments of the described dosage forms can specifically exclude any of the abuse deterrent features described herein.

As for additional abuse deterrent features, the present dosage forms can optionally include a nasal irritant to discourage or prevent abuse by nasal insufflation. The nasal irritant can be a mucous membrane irritant or nasal passageway irritant that, if inhaled through a nasal passageway when contained in a ground or powdered dosage form, can induce pain or irritation of the abuser's nasal passageway tissue. Examples include surfactants such as sodium lauryl sulfate, poloxamer, sorbitan monoesters, and glyceryl monooleates. Certain particular embodiments of dosage forms of the present description do not require, and can specifically exclude, nasal irritant agents such as those described above.

The present dosage forms can include an emetic agent, to cause vomiting. Certain particular embodiments of dosage forms of the present description do not require and can specifically exclude an emetic agent.

The present dosage forms may also or alternatively include one or more agents that exhibit effervescent properties upon exposure to an aqueous medium and that acts as a deterrent to abuse by nasal insufflation. In some embodiments, a gas, e.g., oxygen or carbon dioxide, is released upon exposure of the dosage form to an acidic medium (e.g., less than pH 7, less than pH 6, less than pH 5 or less than pH 4). In some embodiments, the effervescent agent is a base, for example, a carbonate or bicarbonate or a combination thereof. In preferred aspects, the base is sodium bicarbonate. In such embodiments, when the dosage form is exposed to the acidic, aqueous medium, effervescence will occur.

In one exemplary embodiment, the present oral dosage forms represent compressed tablets and comprise
(i) a first population of core-shell particles, each of the core-shell particles of the first population comprises a core that includes a gelling polymer that is hydroxypropylmethycellulose and a wax that is glyceryl behenate; an active pharmaceutical layer surrounding the core, the active pharmaceutical layer comprising esketamine; and at least one layer surrounding the active pharmaceutical layer, the at least one layer comprising a pH-sensitive film comprising Eudragit E 100; and,
(ii) a matrix comprising a carbomer gelling polymer, and sodium bicarbonate, wherein the carbomer gelling polymer and sodium bicarbonate are present in said dosage form in a ratio by weight percentage of about 2:2 based on the total weight of the dosage form, and optionally one or more of a disintegrant, a filler, or a binder;
wherein the dosage form exhibits an immediate release profile of the esketamine when administered to a human in therapeutic doses, and an extended release profile of the esketamine when administered to a human in supratherapeutic doses.

In a further embodiment, the present oral dosage forms represent compressed tablets and comprise
(i) a first population of core-shell particles, each of the core-shell particles of the first population comprises a core that includes a gelling polymer that is hydroxypropylmethycellulose and a wax that is glyceryl behenate; an active pharmaceutical layer surrounding the core, the active pharmaceutical layer comprising esketamine; and at least one layer surrounding the active pharmaceutical layer, the at least one layer comprising a pH-sensitive film comprising Eudragit E 100; and,
(ii) a matrix comprising a carbomer gelling polymer, and sodium bicarbonate, wherein the carbomer gelling polymer and sodium bicarbonate are present in said dosage form in a ratio by weight percentage of about 2:2 based on the total weight of the dosage form, and optionally one or more of a disintegrant, a filler, a glidant, a lubricant, or a binder;
wherein the dosage form exhibits an immediate release profile of the esketamine when administered to a human in therapeutic doses, and an extended release profile of the esketamine when administered to a human in supratherapeutic doses

In a further embodiment, the present oral dosage forms represent compressed tablets and comprise
(i) a first population of core-shell particles, each of the core-shell particles of the first population comprises a core that includes a gelling polymer that is hydroxypropylmethycellulose and a wax that is glyceryl behenate; an active pharmaceutical layer surrounding the core, the active pharmaceutical layer comprising esketamine and hydroxypropylmethycellulose; and at least one layer surrounding the active pharmaceutical layer, the at least one layer comprising a pH-sensitive film comprising Eudragit E 100 and magnesium stearate; and,
(ii) a matrix comprising crospovidone, mannitol, microcrystalline cellulose, silicon dioxide, magnesium stearate, a carbomer gelling polymer, and sodium bicarbonate, wherein the carbomer gelling polymer and sodium bicarbonate are present in said dosage form in a ratio by weight percentage of about 2:2 based on the total weight of the dosage form;
wherein the dosage form exhibits an immediate release profile of the esketamine when administered to a human in therapeutic doses, and an extended release profile of the esketamine when administered to a human in supratherapeutic doses.

In a further exemplary embodiment, the present oral dosage forms represent compressed tablets and comprise
(i) a first population of core-shell particles, each of the core-shell particles of the first population comprises a core that includes a gelling polymer that is hydroxypropylmethycellulose and a wax that is glyceryl behenate; an active pharmaceutical layer surrounding the core, the active pharmaceutical layer comprising esketamine; and at least one layer surrounding the active pharmaceutical layer, the at least one layer comprising a pH-sensitive film comprising Eudragit E 100;
(ii) a second population of core-shell particles that do not include an active pharmaceutical layer;
and,
(iii) a matrix comprising a disintegrant, a filler, a binder, a carbomer gelling polymer, and sodium bicarbonate, wherein the carbomer gelling polymer and sodium bicarbonate are present in said dosage form in a ratio by weight percentage of about 2:2 based on the total weight of the dosage form;
wherein the dosage form exhibits an immediate release profile of the esketamine when administered to a human in therapeutic doses, and an extended release profile of the esketamine when administered to a human in supratherapeutic doses.

In yet another embodiment, the present oral dosage forms represent compressed tablets and comprise
(i) a first population of core-shell particles, each of the core-shell particles of the first population comprises a core that includes a gelling polymer that is hydroxypropylmethycellulose and a wax that is glyceryl behenate; an active pharmaceutical layer surrounding the core, the active pharmaceutical layer comprising esketamine and hydroxypropylmethycellulose; and at least one layer surrounding the active pharmaceutical layer, the at least one layer comprising a pH-sensitive film comprising Eudragit E 100 and magnesium stearate;
(ii) a second population of core-shell particles that do not include an active pharmaceutical layer; and,
(iii) a matrix comprising crospovidone, mannitol, microcrystalline cellulose, silicon dioxide, magnesium stearate, a carbomer gelling polymer, and sodium bicarbonate, wherein the carbomer gelling polymer and sodium bicarbonate are present in said dosage form in a ratio by weight percentage of about 2:2 based on the total weight of the dosage form;
wherein the dosage form exhibits an immediate release profile of the esketamine when administered to a human in therapeutic doses, and an extended release profile of the esketamine when administered to a human in supratherapeutic doses.

In still another embodiment, the present oral dosage forms represent compressed tablets and comprise
(i) a first population of core-shell particles, each of the core-shell particles of the first population comprises a core that includes a gelling polymer that is hydroxypropylmethycellulose and a wax that is glyceryl behenate; an active pharmaceutical layer surrounding the core, the active pharmaceutical layer comprising esketamine and hydroxypropylmethycellulose; and at least one layer surrounding the active pharmaceutical layer, the at least one layer comprising a pH-sensitive film comprising Eudragit E 100 and magnesium stearate;
(ii) a second population of core-shell particles that do not include an active pharmaceutical layer, wherein each of the core-shell particles of the second population comprises a core that includes a gelling polymer that is hydroxypropylmethycellulose and a wax that is glyceryl behenate, and a shell that includes a pH-sensitive film comprising Eudragit E 100 and magnesium stearate; and,
(iii) a matrix comprising crospovidone, mannitol, microcrystalline cellulose, silicon dioxide, magnesium stearate, a carbomer gelling polymer, and sodium bicarbonate, wherein the carbomer gelling polymer and sodium bicarbonate are present in said dosage form in a ratio by weight percentage of about 2:2 based on the total weight of the dosage form;
wherein the dosage form exhibits an immediate release profile of the esketamine when administered to a human in therapeutic doses, and an extended release profile of the esketamine when administered to a human in supratherapeutic doses.

Also disclosed herein are methods for reducing the potential for abuse by a human of an active pharmaceutical ingredient comprising esketamine by simultaneous oral ingestion of multiple dosage units comprising the active pharmaceutical ingredient, comprising providing to the human a dosage form according to any of the embodiments described herein.

Also provided are methods for treating or preventing pain or discomfort in a subject in need thereof by administering to the subject a dosage form according to any of the embodiments described herein. Likewise, the present disclosure provides methods for treating depression in a subject in need thereof by administering to the subject a dosage form according to any of the embodiments described herein.

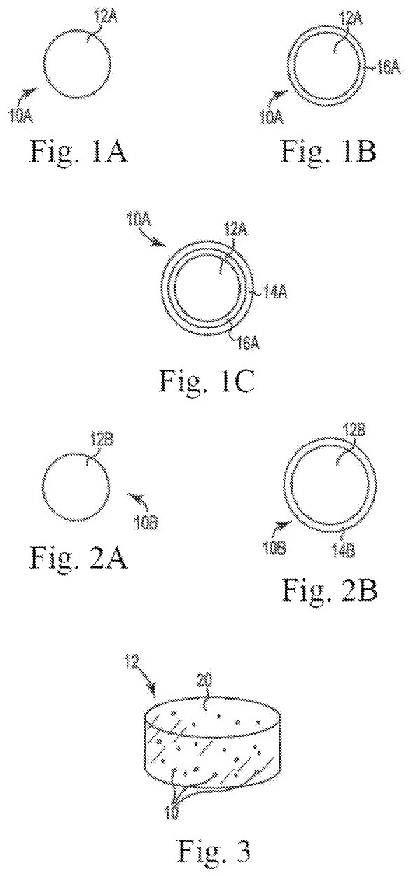

Referring to FIGS. 1A and 1B, a dosage form can include particles 10A that contain esketamine. The particle (e.g., coated particle or “core-shell” particle) can include a core 12A (or “uncoated core”), which may be coated with one or more layers, films or coatings, e.g., 14a, 16a, or any additional layer or coating that is coated over, underneath, or intermediate to these. In FIGS. 1B and 1C, the layer designated 16A may be a drug-containing layer, and the layer designated as 14A may be a solvent resistant, e.g., a pH sensitive film layer. Particle 10A can contain one or more of the ingredients described herein, such as esketamine, a gelling polymer, optional wax, optional solvent-resistant layer, as well as one or more additional layer or layers under, over, or intermediate to these layers or between either layer and the core. Each layer can be present in size or amount (e.g., thickness) that will result in a useful immediate release dosage form having one or more of the presently described abuse deterrent features. Other optional components of a core or layer of particle 10A can be filler, binder, other excipient, or solvent (not more than a residual amount, if any) such as water or ethanol for use in preparing the coated particle, and that is substantially removed after formation of the core, coating, or coated particle. Examples of the core 10A can include any amount or combination of the different ingredients of: a gelling polymer (e.g., from 0 to 100 percent of a core), filler as described herein such as sugar (mannitol) or microcrystalline cellulose (e.g., from 0 to 100 percent of a core), binder (e.g., from 0 to 100 percent of a core), and wax (e.g., from 0 to 100 percent of a core).

While the present dosage forms containing core-shell particles 10A are new and inventive, certain method steps useful to prepare these particles may be known. Available methods include certain methods and processing steps known to be useful for preparing particles and coated particles in the pharmaceutical arts. A core-shell particle 10A can be prepared by an initial step of mixing ingredients of core 12A with a solvent such as water or ethanol and forming the mixture into a spherical core particle by known methods. The particle may be dried and separated by size, and then one or more coating in the form of a continuous film or layer can be applied to the core, optionally successively to produce multiple layers surrounding the core. General processing to produce a multi-layer coated particle can include a series of steps such as compounding, mixing, granulation, wet milling, coating (by any method such as fluidized bed coating, spray coating, etc.), and one or more drying steps such as by use of a fluidized bed or other drying method. Intermittently between core-forming and coating steps, e.g., after a drying step, coated or uncoated particles can be sorted or separated based on size to produce a composition or a collection of particles having a desired size range and distribution. Accordingly, coated granulate compositions according to the invention may be prepared by a process comprising:
(i) granulating a wax or a gelling polymer, or a mixture thereof, in the presence of a hydroalcoholic solution or suspension comprising a suitable binder, to form granules;
(ii) layering the granules formed in step (i) with a solution or suspension comprising esketamine; and
(iii) coating the layered granules formed in step (ii) with a solution or suspension comprising a film forming polymer material to form a coated layered granulate.
   The process above may further comprise steps of milling and drying the granulate formed in step (i).

In instances wherein the core comprises a sugar sphere or a microcrystalline cellulose sphere, the steps of the process above would be modified as follows:
(i) providing a sugar sphere (or microcrystalline cellulose sphere);
(ii) layering the sugar sphere (or microcrystalline cellulose sphere) with a solution or suspension comprising an API; and
(iii) coating the layered sphere formed in step (ii) with a solution or suspension comprising a film forming polymer material to form a coated layered sphere.

Compressed tablets according to the invention may be prepared by a process comprising:
(i) combining the coated layered granulate (or the coated layered sphere) prepared according to either of the above processes with a second API (e.g., acetaminophen), a gelling polymer, and a disintegrant, and optionally, with at least one additional excipient selected from a filler, a colorant, and a pH adjusting agent, to form a first mixture and then blending the first mixture for a suitable time;
(ii) adding a lubricant to the blended mixture formed in step (i) to form a second mixture, and then blending the second mixture for a suitable time;
(iii) compressing the blended mixture formed in step (ii) to form compressed tablets.

A suitable time for the blending in step (i) may be, for example, from about 5 to about 90 minutes, or from about 10 to about 60 minutes, or from about 20 to about 40 minutes, or about 30 minutes. A suitable time for the blending in step (ii) may be, for example, from about 1 to about 30 minutes, or from about 5 to about 20 minutes, or about 10 minutes.

In certain embodiments as shown at FIGS. 1A, 1B, and 1C, an immediate release dosage form as described can include a core-shell particle 10A that includes a core 12A that contains no drug, only a minor amount of API, or an insubstantial amount of API. Core 12A may contain less than 5 weight percent, e.g., less than 1 or less than 0.5 weight percent active pharmaceutical ingredient based on a total weight of the core of the core-shell particle. Alternatively, core 12A may contain less than 5 weight percent of a total amount of pharmaceutical ingredient in a core-shell polymer, e.g., less than 5, less than 1, or less than 0.5 weight percent active pharmaceutical ingredient based on total weight of API in the core-shell particle. In these embodiments a major portion of API can be contained outside of core 12A, e.g., in an API layer 16A, which can contain at least 50, at least 75, or at least 90, or at least 95 weight percent of a total amount of the API in a core-shell polymer.

Core 12A can include binder, gelling polymer (e.g., HPMC), wax, filler, or any combination thereof, each in an amount to allow the materials of the core to function as one or more abuse deterrent features as described herein. See the examples included herewith for examples of useful amounts and ranges of amounts of these ingredients. In a preferred embodiment, core 12A includes HPMC, glyceryl behenate, and ethylcellulose.

Referring to FIG. 1A, core 12A contains gelling polymer, wax, binder, or filler, or any combination of these, and no API (meaning not more than an insignificant amount, such as less than 0.5, less than 0.1, or zero weight percent based on the weight of core 12A). As shown at FIGS. 1B and 1C, core 12A, not containing API, can be coated with a coating layer that contains API, e.g., an active pharmaceutical layer or API layer 16A. As shown at FIG. 1B, core-shell particle 10A includes core 12A, which does not contain any API, and API layer 16A, which contains an amount of API, such as a total amount of API (e.g., API commonly susceptible to abuse) to be contained in a dosage form prepared from particles 10A. API layer 16A can contain one or more ingredients as described herein useful to form API layer 16A as a layer over an outer surface of core 12A. API in API layer 16A can account for all of or most of (e.g., at least 70, at least 80, at least 90, or at least 95 percent) the total amount of that type of API in the core-shell particles and in the dosage form; in this embodiment, the core can contain less than 10, less than 5, or less than 1 percent of the total amount of API in the core-shell particles, and less than 10, 5, or 1 percent of the total amount of API in the dosage form. Useful non-API ingredients in an API layer can include a binder or a gelling polymer along with the API. The API and non-API ingredients can be carried in a solvent (e.g., water, ethanol, or both) and coated and dried to form a preferably continuous film layer on an outer surface of core 12A, i.e., API layer 16A. See the examples included herewith for examples of useful amounts and ranges of amounts of these ingredients. In a preferred embodiment, the API layer 16A includes esketamine and hypromellose (HPMC).

A core-shell particle 10A can also optionally include a film layer, e.g., a solvent-resistant layer (e.g., a pH-sensitive layer) 14A as described herein.

In certain alternate embodiments a dosage form as described can include a core-shell particle 10B that includes a core 12B that does contain a useful amount of API, such as an amount of API useful in an immediate release dosage form having one or more abuse deterrent features as described herein, prepared to include particles 10B. See FIGS. 2A and 2B. According to such embodiments, core 12B of particle 10B can contain a gelling polymer, optional wax, optional binder or filler, and an amount of API.

Referring to FIG. 2A, core 12B contains gelling polymer, optional wax, optional binder, and API. Referring to FIG. 2B, core 12B, containing API, can optionally be coated with solvent-resistant layer (e.g., a pH-sensitive layer) 14B as described herein for use in an immediate release dosage form. Core 12B may also optionally be coated with a coating layer that contains API, e.g., an active pharmaceutical layer or API layer prior to application of the solvent-resistant layer. Accordingly, API containing core-shell particles as described herein may contain API of a type that is susceptible to abuse:
in an API layer surrounding the core and in a substantial amount in the core;
in an API layer surrounding the core and in an insubstantial amount in the core;
only in an API layer surrounding the core; or
only in the core.

In certain alternative embodiments, a dosage form as described can include a core-shell particle 10B, as depicted in FIG. 2B, that that does not contain an API layer, and does not contain any API. Referring to FIG. 2C, such a particle 10B, containing no API, may include core 12B containing gelling polymer, optional wax, and optional binder, which core 12B may optionally be coated with solvent-resistant layer (e.g., a pH-sensitive layer) 14B as described herein for use in an immediate release dosage form. In a preferred embodiment of a core-shell particle 10B that does not contain an API layer, the core includes Hypromellose, glyceryl behenate, and ethylcellulose.

A coated particle 10A or 10B that includes API, and optionally, a coated particle 10B that does not include API, can be included in any of a variety of dosage forms, examples including a compressed tablet or compressed capsule, a suppository, capsule, caplet, pill, gel, soft gelatin capsule, etc. As one example, a dosage form 12 can be prepared as a compressed tablet or compressed capsule. Tablet or capsule 12 can contain core-shell particles 10 (e.g., 10A or 10B) distributed within a matrix 20, compressed to form the compressed tablet or capsule 12. Core-shell particles 10A or 10B can be as described herein, generally or specifically, and can contain an amount of API suited to provide a desired dosage upon ingestion of tablet or capsule 12; e.g., matrix 20 does not include any substantial amount of API, or contains no API at all.

Matrix 20 can include ingredients useful in combination with the core-shell particles 10A, 10B, to produce an immediate release dosage form. Examples of useful excipients of an immediate release dosage form can include ingredients that allow the dosage form to break up or disintegrate upon ingestion and facilitate exposure to fluid in a stomach, such as a useful amount of disintegrant. Examples of such excipients for such a dosage form can also include one or more ingredients that act as an abuse deterrent feature, such as a gelling polymer as described herein. Other excipients can be useful for processing to form a compressed dosage form, and also may allow the compressed dosage form to function as an immediate release dosage form, with one or more abuse deterrent features. In certain embodiments, the matrix includes a filler, a disintegrant, a binder, a gelling polymer, a pH adjuster, a glidant, and a lubricant.

The following non-limiting examples show various dosage forms as described herein. The described and exemplified dosage forms can be made from methods that include granulating, coating, and compressing steps as follows.

### Example 1—General Procedure for Preparation of Tablet Dosage Form

### Granulation

1. Glyceryl behenate and hypromellose K100M were dry mixed in a high shear granulator. Hydroalcoholic solution of ethylcellulose was added. Alternatively, the granulation can be produced through top spraying the hydroalcoholic solution in a fluid bed granulator. Optionally, a portion of the ethyl cellulose, for example from about 10 to about 50% by weight, or from about 10 to about 40% by weight, or from about 15 to about 30% by weight, may be dry mixed with the glyceryl behenate and hypromellose K100M prior to adding the hydroalcoholic solution containing the balance of the ethyl cellulose.
Alternative approach when API is included in the core: Glyceryl behenate and hypromellose K100M and API are dry mixed in a high shear granulator. Hydroalcoholic solution of ethylcellulose is added. Alternatively, the granulation can be produced through top spraying the hydroalcoholic solution in a fluid bed granulator. Optionally, a portion of the ethyl cellulose, for example from about 10 to about 50% by weight, or from about 10 to about 40% by weight, or from about 15 to about 30% by weight, is dry mixed with the glyceryl behenate and hypromellose K100M prior to adding the hydroalcoholic solution containing the balance of the ethyl cellulose.

2. The granules were then wet milled using a size reduction mill (Granumill) and then dried using a fluid bed, and optionally screened.

### Layering

3. The polymer granules were then layered using Wurster fluid bed layering process with esketamine and hypromellose K100M (or alternatively, granulated using high shear granulation or top spray fluid bed granulation process).
Alternative approach when API is not in the coated granule: the layering step is omitted and the coating of Step 4 below is applied to the granulate prepared in Step 1.

### Coating

4. The layered granules of Step 3 (or alternatively, when the coated granule will not contain API, the granules prepared in Step 1) were then coated using a fluid bed coater equipped with a Wurster insert (bottom spray assembly) with ethanolic suspension of Eudragit E100 copolymer and magnesium stearate. Coated particles were then screened and blended.

### Blending and Tablet Compression

The blending, compression and bottling process for esketamine tablets manufactured using the coated intermediate is as follows:
1. The API-containing coated granules, crospovidone, Carbopol® 71G, sodium bicarbonate, mannitol, microcrystalline cellulose, optionally, coated granules containing no API, optionally, a glidant such as colloidal silicon dioxide, and optionally a desired colorant, were then added to the blender and mixed.
2. Magnesium stearate (and optionally colorant) was then added to the blender and mixed. The blend was compressed into tablets using a rotary tablet press.

### Example 2—Preparation of Polymer Granules

Granules were manufactured in a high shear granulator, where hypromellose and glyceryl behenate were dry mixed for 3 minutes. Then, a 10% hydroalcoholic solution of ethylcellulose N10 was slowly added while maintaining the granulator impeller and chopper speed at pre-selected values that provide enough shear for granule formation and growth. Solution addition was continued until the entire amount of ethylcellulose was added. The granules were then wet milled using a size reduction mill (Granumill) and were subsequently loaded into fluid bed for drying. Table 1, below, provides the relative proportions of the reagents used for forming the granules.

TABLE 1
Components for granule formulation

| Component | % w/w |
|---|---|
| Hypromellose K100M | 60.09 |
| Glyceryl behenate | 25.75 |
| Ethylcellulose N-10 | 14.16 |
| TOTAL | 100 |

The prepared granules were then layered in a bottom spray fluid bed coater with an aqueous coating dispersion esketamine HCl and hypromellose 2910.

TABLE 2
Components for coating dispersion used
for coating of polymer granules

| | | g/Batch, | g/Batch, |
|---|---|---|---|
| Component within | | 19.5% | 39.1% |
| Coating Dispersion | % w/w | Esketamine | Esketamine |
| Esketamine HCl | 13 | 195 | 390 |
| Hypromellose 2910 | 5 | 75 | 150 |
| Purified Water | 82 | 1260 | 2520 |
| TOTAL | 100 | 1500 | 3000 |

TABLE 3
Components for layered granule formulation,
19.5% or 39.1% Esketamine HCl

| | | g/Batch, | g/Batch, |
|---|---|---|---|
| | | 19.5% | 39.1% |
| Layered Granule | % w/w | Esketamine | Esketamine |
| Polymer granules from Table 1 | 68.83 | 596.1 | 325.4 |
| Solids from Coating | 31.17 | 270 | 540 |
| Dispersion from Table 2 | | | |
| TOTAL | 100 | 866.1 | 865.4 |

The esketamine HCl layered granules were then coated in a bottom spray fluid bed coater with the alcoholic suspension of Eudragit E-100 copolymer and magnesium stearate. The resulting coated granules (containing either 16% or 32% esketamine HCl) were subsequently used for further blending and compression process.

TABLE 4
Components for coating dispersion used for
coating of Esketamine HCl layered granules

| | | g/Batch, | g/Batch, |
|---|---|---|---|
| Component within | | 19.5% | 39.1% |
| Coating Dispersion | % w/w | Esketamine | Esketamine |
| Eudragit E100 | 16.67 | 102 | 102 |
| Magnesium Stearate | 8.33 | 51 | 51 |
| Alcohol USP | 75 | 489 | 489 |
| TOTAL | 100 | 612 | 612 |

TABLE 5
Components for coated esketamine granules
containing 16% Esketamine HCl

| Component | % w/w |
|---|---|
| Esketamine HCl layered granules, 19.5% | 82 |
| Solids from coating dispersion from Table 4 | 18 |
| TOTAL | 100 |

TABLE 6
Components for coated esketamine granules
containing 32% Esketamine HCl

| Component | % w/w |
|---|---|
| Esketamine HCl layered granules, 39.1% | 82 |
| Solids from coating dispersion from Table 4 | 18 |
| TOTAL | 100 |

Coated polymer granules that did not contain esketamine HCl were formed using the polymer granules of Table 1.

In order to form the coated polymer granules, polymer granules of Table 1 were coated in a bottom spray fluid bed coater with the alcoholic suspension of Eudragit E-100 copolymer and magnesium stearate (Table 7). The resulting coated granules were, along with the coated esketamine granules, subsequently used for further blending and compression process.

TABLE 7
Components for coating dispersion used
for coating of polymer granules

| Component within | | |
|---|---|---|
| Coating Dispersion | % w/w | g/Batch |
| Eudragit E100 | 16.67 | 366 |
| Magnesium Stearate | 8.33 | 182.8 |
| Alcohol USP | 75 | 1676 |
| TOTAL | 100 | 2194.8 |

TABLE 8
Components for coated polymer granules (no esketamine)

| Component | % w/w |
|---|---|
| Polymer granules from Table 1 | 50 |
| Solids from coating dispersion from Table 7 | 50 |
| TOTAL | 100 |

### Example 3—Esketamine HCl Tablet Formulation

The coated granules prepared according to Example 2, supra, were mixed with excipients as listed in Tables 9-13, below, and blended in a V-blender for 30 minutes. Both coated esketamine granules (Tables 4/5 or 4/6) and coated polymer granules (Tables 7/8) were included as indicated. Magnesium stearate was then added to lubricate the blend and the mixture was blended for an additional 5 minutes prior to compressing into esketamine HCl tablets.

TABLE 9
Tablets, 100 mg Esketamine

| | % w/w |
|---|---|
| Coated Esketamine Granules, 33% | 30.77 |
| Coated Polymer Granules from Tables 7/8 | 17.6 |
| Mannitol (Mannogem EZ) | 13.39 |
| Crospovidone (Polyplasdone XL) | 20 |
| Microcrystalline Cellulose (Avicel PH113) | 13 |
| Carbomer (Carbopol ® 71G) | 2 |
| Sodium Bicarbonate | 2 |
| Colloidal silicon dioxide | 0.2 |
| Magnesium stearate | 1 |
| Total | 100 |

TABLE 10
Tablets, 40 mg Esketamine

| | % | mg/Tab | g/batch |
|---|---|---|---|
| Coated Esketamine Granules, 16.0% | 25.00 | 250.0 | 2500 |
| Coated Polymer Granules | 7.06 | 70.6 | 706 |
| Mannitol (Mannogem EZ) | 29.74 | 297.4 | 2974 |
| Crospovidone (Polyplasdone XL) | 20 | 200 | 2000 |
| Microcrystalline Cellulose (Avicel PH113) | 13 | 130 | 1300 |
| Carbomer (Carbopol ® 71G) | 2 | 20 | 200 |
| Sodium Bicarbonate | 2 | 20 | 200 |
| Colloidal silicon Dioxide | 0.2 | 2 | 20 |
| Magnesium stearate | 1 | 10 | 100 |
| Total | 100 | 1000 | 10000 |

TABLE 11
Tablets, 20 mg Esketamine

| | % | mg/Tab | g/batch |
|---|---|---|---|
| Coated Esketamine Granules, 16.0% | 12.5 | 125 | 1250* |
| Coated Polymer Granules | 21.17 | 211.7 | 2117* |
| Mannitol (Mannogem EZ) | 28.13 | 281.3 | 2813 |
| Crospovidone (Polyplasdone XL) | 20 | 200 | 2000 |
| Microcrystalline Cellulose (Avicel | 13 | 130 | 1300 |
| PH113) | | | |
| Carbomer (Carbopol ® 71G) | 2 | 20 | 200 |
| Sodium Bicarbonate | 2 | 20 | 200 |
| Colloidal silicon Dioxide | 0.2 | 2 | 20 |
| Magnesium stearate | 1 | 10 | 100 |
| Total | 100 | 1000 | 10000 |
| *Polymer granules required | | | 1766 |
| | | | (707 + 1059) |

TABLE 12
Tablets, 10 mg Esketamine

| | % | mg/Tab | g/batch |
|---|---|---|---|
| Coated Esketamine Granules, 16.0% | 6.25 | 62.5 | 625* |
| Coated Polymer Granules | 27.42 | 274.2 | 2742* |
| Mannitol (Mannogem EZ) | 28.13 | 281.3 | 2813 |
| Crospovidone (Polyplasdone XL) | 20 | 200 | 2000 |
| Microcrystalline Cellulose (Avicel | 13 | 130 | 1300 |
| PH113) | | | |
| Carbomer (Carbopol ® 71G) | 2 | 20 | 200 |
| Sodium Bicarbonate | 2 | 20 | 200 |
| Colloidal silicon Dioxide | 0.2 | 2 | 20 |
| Magnesium stearate | 1 | 10 | 100 |
| Total | 100 | 1000 | 10000 |
| *Polymer granules required | | | 1725 |
| | | | (354 + 1371) |

TABLE 13
Tablets, 5 mg Esketamine

| | % | mg/Tab | g/batch |
|---|---|---|---|
| Coated Esketamine Granules, 16.0% | 3.125 | 31.25 | 312.5* |
| Coated Polymer Granules | 30.545 | 305.45 | 3054.5* |
| Mannitol (Mannogem EZ) | 28.13 | 281.3 | 2813 |
| Crospovidone (Polyplasdone XL) | 20 | 200 | 2000 |
| Microcrystalline Cellulose (Avicel ® | 13 | 130 | 1300 |
| PH113) | | | |
| Carbomer (Carbopol ® 71G) | 2 | 20 | 200 |
| Sodium Bicarbonate | 2 | 20 | 200 |
| Colloidal silicon Dioxide | 0.2 | 2 | 20 |
| Magnesium stearate | 1 | 10 | 100 |
| Total | 100 | 1000 | 10000 |
| *Polymer granules required | | | 1704 |
| | | | (177 + 1527) |

### Example 4—Preparation of Comparative Embodiment

Comparative 100 mg esketamine HCl embodiments having a matrix containing carbomer gelling polymer and sodium bicarbonate in a ratio of 2:10 percentage by weight, based on the total weight of the dosage form.

Esketamine HCl layered granules were coated in a bottom spray fluid bed coater with the alcoholic suspension of Eudragit E-100 copolymer and magnesium stearate. The resulting coated granules (containing 33% esketamine HCl) were subsequently used for further blending and compression process.

The coated granules were mixed with excipients as listed in Table 14, below, and blended in a V-blender for 30 minutes. Magnesium stearate was then added to lubricate the blend and the mixture was blended for an additional 5 minutes prior to compressing into esketamine HCl tablets.

TABLE 14
100 mg esketamine tablet formulations with
2:10 carbomer to sodium bicarbonate ratio

| | % w/w |
|---|---|
| Coated Esketamine Granules, 33% | 30.77 |
| Coated Polymer Granules from Tables 7/8 | 17.6 |
| Mannitol (Mannogem EZ) | 5.37 |
| Crospovidone (Polyplasdone XL) | 20 |
| Microcrystalline Cellulose (Avicel PH113) | 13 |
| Carbomer (Carbopol ® 71G) | 2 |
| Sodium Bicarbonate | 10 |
| Colloidal silicon dioxide | 0.2 |
| Magnesium stearate | 1 |
| Red Iron Oxide | 0.06 |
| Total | 100 |

### Example 5—Evaluation of Esketamine Release

Tablet dosage forms according to the present disclosure (100 mg esketamine HCl—see Table 9, supra) were tested for single-tablet dissolution by depositing a single tablet into a USP II apparatus containing 500 mL of deaerated 0.01N hydrochloric acid medium and a paddle speed set at 50 RPM.

Also tested were comparative 100 mg esketamine HCl embodiments having a matrix containing carbomer gelling polymer and sodium bicarbonate in a ratio of 2:10 percentage by weight, based on the total weight of the dosage form, that were prepared as described in Example 4, supra.

FIG. 4 shows the results of single-tablet dissolution testing of the inventive 2:2 dosage form (triangles) and the comparative 2:10 dosage form (squares). Both embodiments displayed complete release within 15 minutes following deposition into the testing apparatus.

### Example 6—Evaluation of Gel Characteristics

An evaluation was conducted to assess the quality of the gels that were formed when the inventive 2:2 weight percent dosage form and the comparative 1:2 and 2:10 weight percent dosage forms were exposed to dissolution medium.

In particular, a dose consisting of 10 tablets of the inventive 2:2 weight percent dosage form of Example 3 (Table 9) was placed into an aqueous medium of 0.025 N HCl+70 mM NaCl at 37° C. The medium also contained a dual mesh screen featuring a horizontally-oriented circular top screen, a horizontally-oriented circular bottom screen, and a handle for lifting the dual mesh apparatus from the medium. Fifteen minutes after mixing the deposited supratherapeutic dose into the test medium, the dual mesh apparatus was removed from the medium and the characteristics of the gel were visually assessed.

The same procedure was performed with respect to a dose of 10 tablets of the comparative 1:2 and 2:10 dosage forms of Example 4 and Table 15, below, and the characteristics of the resulting gel were assessed and compared to the gel resulting from the dose of the 2:2 inventive dosage form.

TABLE 15
100 Mg Esketamine Tablet Formulations With
1:2 Carbomer To Sodium Bicarbonate Ratio

| | % w/w |
|---|---|
| Coated Esketamine Granules, 32% | 29.94 |
| Coated Polymer Granules from Tables 7/8 | 16.82 |
| Mannitol (Mannogem EZ) | 16.04 |
| Crospovidone (Polyplasdone XL) | 20 |
| Microcrystalline Cellulose (Avicel PH113) | 13 |
| Carbomer (Carbopol ® 71G) | 1 |
| Sodium Bicarbonate | 2 |
| Colloidal silicon dioxide | 0.2 |
| Magnesium stearate | 1 |
| Total | 100 |

All samples formed gels in under 15 minutes. Visual inspection of the dual screen apparatus that was lifted from the medium into which the dose of the 2:2 inventive dosage form was tested revealed that the gel from the dose (i) was retained on the upper surface of the top screen in a very thin layer; (ii) was retained on the lower surface of the top screen in a thin layer; (iii) was retained on the upper surface of the bottom screen in a robust layer that was at least twice as thick (vertically) as the layer that was retained on the lower surface of the top screen; and, (iv) overall, represented a good quality gel that was not overly rigid, nor was it watery (was retained on the bottom screen and did not flow through the screen when the apparatus was lifted from the medium). FIG. 5 provides an image of the dual screen apparatus that was used to test the supratherapeutic dose of the 2:2 inventive dosage form with gel retained on the screens as described above.

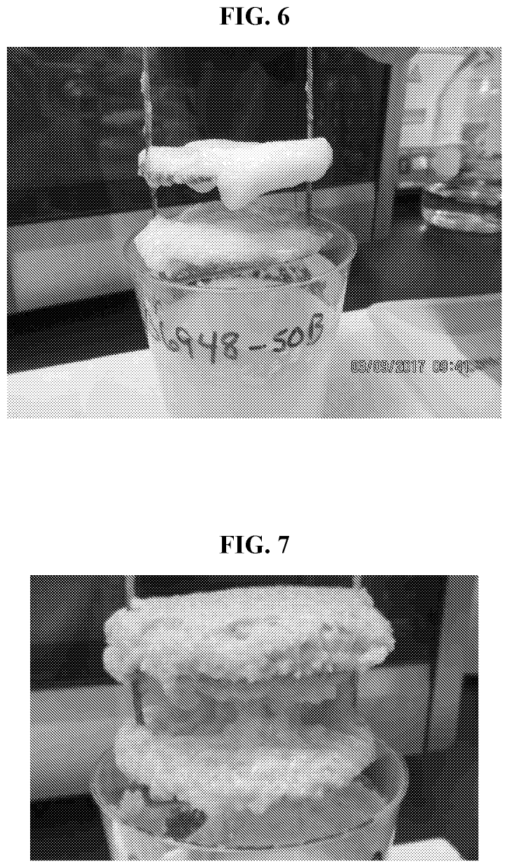

Visual inspection of the dual screen apparatus that was lifted from the medium into which the dose of the 1:2 comparative dosage form was tested revealed that the gel from the dose (i) was retained on the upper surface of the top screen in a weak layer that was thinner than a layer that was retained on the upper surface of the bottom screen; (ii) was retained on the lower surface of the top screen in a thin layer; (iii) was not retained between the top and bottom screens; and, (iv) appeared to flow through the screens as a weak gel. FIG. 6 provides an image of the dual screen apparatus that was used to test the dose of the 1:2 inventive dosage form with gel retained on the screens as described above.

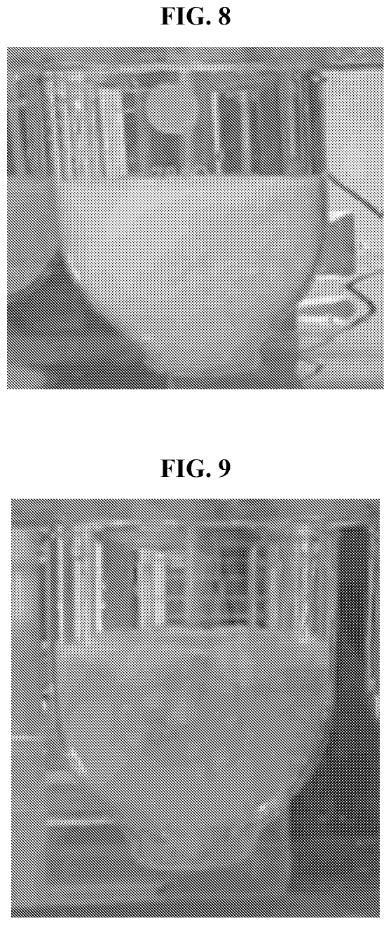

Visual inspection of the dual screen apparatus that was lifted from the medium into which the dose of the 2:10 comparative dosage form was tested revealed that the gel from the dose (i) was retained on the upper surface of the top screen in a robust layer that was thicker than a layer that was retained on the upper surface of the bottom screen; (ii) was retained on the lower surface of the top screen in a thin layer; (iii) was retained on the upper surface of the top screen in a layer that was not as thick as the layer that was retained on the upper surface of the top screen; (iv) included “runners” (portions of gel) between the layer of gel on the lower surface of the top screen and the layer of gel on the upper surface of the bottom screen, and, (v) overall, represented a rigid, thick gel. FIG. 7 provides an image of the dual screen apparatus that was used to test the dose of the 2:10 comparative dosage form with gel retained on the screens as described above.

A further evaluation was conducted as to the quality of the gels that were formed when respective doses of the inventive 2:2 dosage form and the comparative 2:10 dosage form were exposed to dissolution medium.

A dose consisting of 10 tablets of the inventive 2:2 dosage forms of Example 3 (Table 9) was placed into an aqueous medium of 0.025 N HCl+70 mM NaCl at 37° C. The same procedure was performed in a second test vessel with respect to a dose of 10 tablets of the comparative 2:10 dosage form of Example 4 (Table 14), and the characteristics of the material within the vessel were assessed and compared to the material within the vessel containing the dose of the 2:2 inventive dosage form.

FIG. 8 provides an image of the gel resulting from the dose of the inventive 2:2 dosage forms, suspended in the test medium. As shown, the gel/test medium mixture appeared turbid and opaque, indicating that the gel material was substantially homogeneously dispersed within the medium. There were no uneven concentrations of gel in any particular region of the medium (e.g., in the upper, middle, or lower portions of the medium within the test vessel), such as in the form of clumps, and likewise no regions without association between medium and gel, wherein medium is clearly visible in the absence of gel material.

FIG. 9 provides an image of the gel resulting from the dose of the comparative 2:10 dosage forms, suspended in the test medium. As shown, the gel/test medium mixture was non-uniform, and included a concentration of gel material within the upper portion of the medium, indicating that the gel material was not uniformly dispersed within the medium. In fact, gel material was absent from a significant volume of the medium. Also apparent were clumps of gel and portions of medium that were clearly visible in the absence of gel material.

### Example 7—Physical Manipulation

Initial analysis. A single 40 mg tablet of esketamine HCl of example 3 was crushed using a mortar and pestle. The tablet was initially fractured by the gentle striking of the pestle, followed by rotation of the pestle against the tablet pieces. After visual inspection showed that no further particle size reduction was possible and the procedure terminated. The sample was then collected onto a piece of (pre-tared on an analytical balance) 3×3 weigh paper by gently brushing the material from the mortar and weighed. 1000 μm and 500 μm sieves were stacked sequentially and set in a sieve shaker following which, the samples was transferred from the weigh paper by distributing it across the top sieve (1000 μm). Each sample was sieved for a total of 5 minutes during which for the first 60 seconds, the shaker was set to an amplitude of 80 Hz, and for the following 4 minutes, at an amplitude of 50 Hz. After sieving, each sieve was carefully removed and the weight of the sieve and manipulated material was recorded. The material from each sieve was then transferred into a tarred and labeled collection vessel and weighed. The procedure was repeated in triplicate and the results averaged to show an average manipulation time of 52 seconds, 99.7% weight recovery, 0.2% particle size above 1000 μm and 3.0% particle size between 500 and 1000 μm.

Advanced analysis. A single 40 mg tablet of esketamine HCl of example 3 was crushed using a mortar and pestle. The tablet was initially fractured by the gentle striking of the pestle, followed by rotation of the pestle against the tablet pieces for 52 seconds. The sample was then collected onto a piece of (pre-tared on an analytical balance) 3×3 weigh paper by gently brushing the material from the mortar and weighed. 1000 μm, 500 μm, 212 μm, 106 μm and 75 μm sieves were stacked sequentially and set in a sieve shaker following which, the samples was transferred from the weigh paper by distributing it across the top sieve (1000 μm). Each sample was sieved for a total of 5 minutes during which for the first 60 seconds, the shaker was set to an amplitude of 80 Hz, and for the following 4 minutes, at an amplitude of 50 Hz. After sieving, each sieve was carefully removed and the weight of the sieve and manipulated material was recorded. The material from each sieve was the collected onto a piece of (pre-tared on an analytical balance) 3×3 weigh paper by gently brushing the material from the sieve and weighed. Sample fractions that resulted in 4% recovery or less were transferred into 20 mL scintillation vials, fractions that resulted in between 4% and 25% recovery were transferred into 125 mL Erlenmeyer Flasks and fractions that resulted in over 25% recovery were transferred into 250 mL Erlenmeyer Flasks. To each vessel with collected material, a calculated prorated volume of 0.1 N HCl (mass recovered on sieve]/[mass of manipulated material]*240 mL) was added. The sieve fraction solutions were then extracted overnight for a minimum of 15 hours at room temperature with agitation at 150 RPM following which, aliquots of the samples were prepared for LC-MS/MS analysis. The procedure was repeated in triplicate and the results averaged to show an average 98.8% weight recovery. The results per sieve fraction are shown in Table 16, below.

TABLE 16
Summary of results per sieve fraction

| | | Average % | Average % | Average % |
|---|---|---|---|---|
| | | Particle Size | esketamine | esketamine |
| Sieve fraction | | Distribution | recovery | uniformity |
| >1000 | μm | 0.3 | 0.1 | 0.8 |
| 500-1000 | μm | 5.4 | 9.7 | 7.4 |
| 212-500 | μm | 31.9 | 62.4 | 8.0 |
| 106-212 | μm | 26.7 | 18.9 | 3.0 |
| 75-106 | μm | 24.3 | 5.2 | 0.9 |
| <75 | μm (pan) | 11.4 | 1.8 | 0.7 |

### Example 8—Visual Analysis

Samples of 40 mg pure esketamine (as base) powder or a 40 mg tablet of esketamine HCl of example 3, either whole or after gentle crushing with a mortar and pestle, were introduced to glass beakers containing 200 ml of a beverage and observed over a 90 second period for changes to the beverage's appearance. After about 30 seconds, the beverage and esketamine were mixed gently using a glass mixing rod until either there was uniformity in the visual appearance of the mixed sample, or the sample effervesced and spilt over the top of the beaker.

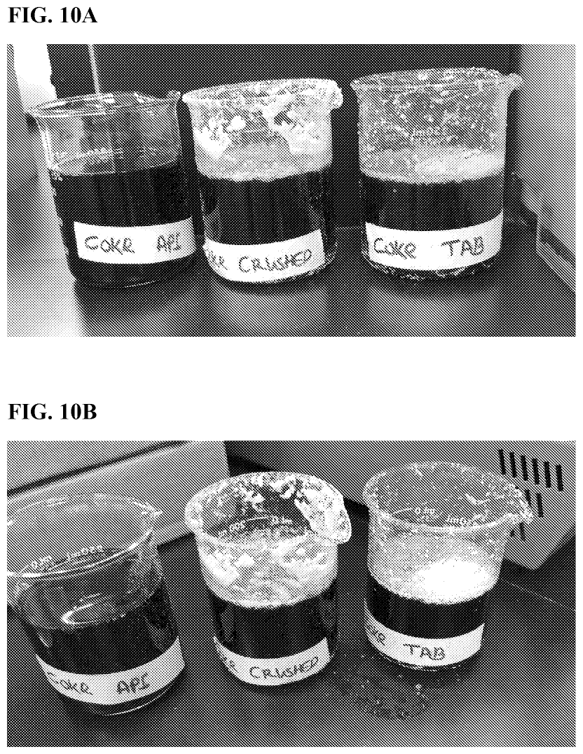

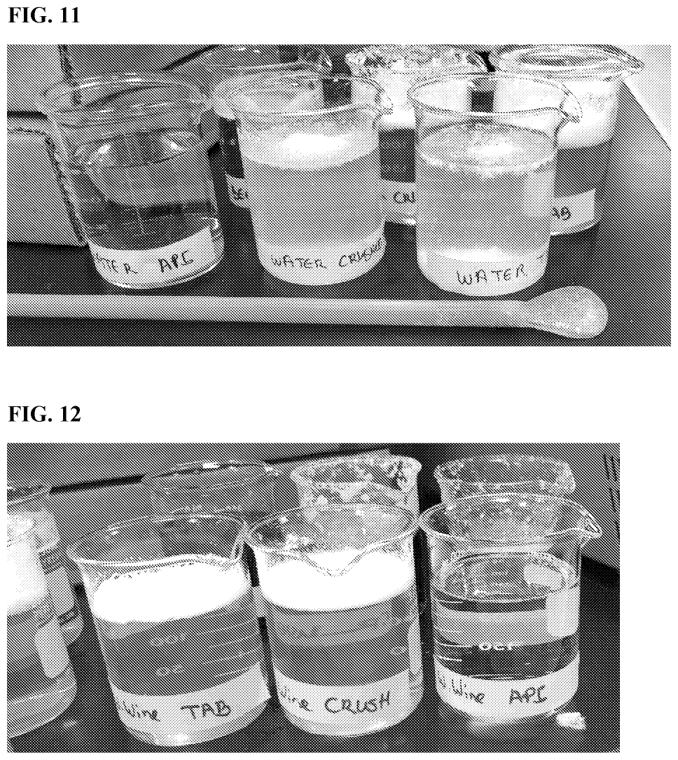

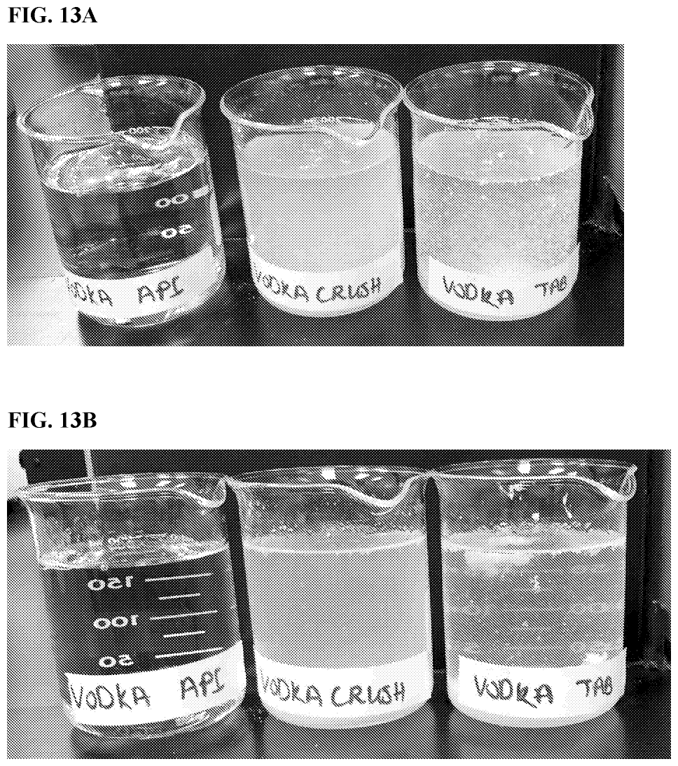

The results of the analysis are shown in Table 17 and images of the sample after 90 seconds shown in FIGS. 10A-13B.

TABLE 17
Summary of visual findings

| | Powdered esketamine | Crushed tablet | Complete tablet |
|---|---|---|---|
| Water | Clear solution | Cloudy dispersion, | Cloudy dispersion, |
| | | particles bouyant on | particles bouyant on |
| | | surface | surface and base of |
| | | | beaker |
| Cola | Clear solution | Strong residue of | Residue of |
| | | efferverscence on | effervescence on |
| | | beaker walls | beaker walls |
| White wine | Clear solution | Fine dispersion, film | Fine dispersion, film |
| | | on beverage surface | on beverage surface |
| Vodka | Clear solution | Cloudy dispersion | Particulate |
| | | | dispersion, particles |
| | | | bouyant on surface |
| Beer | Clear solution | Residue of | Residue of |
| | | efferverscence on | effervescence on |
| | | beaker walls | beaker walls |

### Example 9—Turbidity Analysis

Samples of between 240 ml (unless stated otherwise below) of a variety of beverages, as per the following list are transferred into 2 separate clean pre-labeled 250 mL glass jars. An additional 20 ml of each beverage is transferred into a clean, dry turbidity meter sample cuvette.
1. Bottled Water
2. Cranberry juice cocktail
3. Lemon Lime soda
4. Cola
5. Ginger ale
6. Vodka
7. Vodka Cranberry
8. Vodka Sprite
9. Whiskey *75 mL (approximately 2.5 fl oz.)
10. Whisky Cola (50:50 ratio)
11. Whiskey Ginger ale (50:50 ratio)
12. Light beer
13. Dark beer
14. Red wine *175 mL (approximately 6 fl oz.)
15. White wine *175 mL (approximately 6 fl oz.)

In one of the glass jars, a single crushed 40 mg tablet of esketamine HCl of example 3 is added and stirred with a metal spatula for 1 minute (measured with a traceable timer). 20 mL of this solution is then transferred into a clean, dry turbidity meter sample cuvette. To the remaining (second) glass jar, a single crushed 40 mg tablet of esketamine HCl of example 3 is added and stirred just well enough to wet the manipulated tablet material. 20 mL of this solution is then transferred into a second clean, dry turbidity meter sample cuvette.

The turbidity measurement in Turbidity measurements NTU (Nephelometric Turbidity Units) of the unadulterated beverage and both adulterated beverage samples are recorded using a Lovibond TB 300 IR turbidity meter. The test is repeated in duplicate and the results averaged.

1. An enteral dosage form comprising esketamine and one or more excipients that produce a visual indication of the presence of the dosage form within a vessel containing a liquid beverage, the visual indication including dispersion of visible particles, cloudiness, or both, within the beverage.

2. The dosage form according to claim 1, wherein the visual indication further includes at least a portion of the dosage form that is buoyant on the surface of the beverage, a film at the surface the beverage, a foam resulting from an effervescent reaction between the dosage from and the beverage, residue from the dosage form on an inner surface of the vessel, or any combination thereof.

3. The dosage form according to claim 1, wherein said visual indication includes an absence of a film or foam at an upper surface of the beverage.

4. The dosage form according to claim 1, wherein said visual indication includes a film or foam at an upper surface of the beverage.

5. The dosage form according to claim 1, wherein the indication is visible within the beverage for at least 2 minutes, 5 minutes, 7 minutes, 10 minutes, 12 minutes, 15 minutes, 20 minutes, 25 minutes, 30 minutes, 40 minutes, 50 minutes, or 1 hour following dispersion within the beverage, in the absence of deliberate removal thereof.

6. The dosage form according to claim 1, wherein the visual indication comprises increasing the turbidity of the beverage, exclusive of any concentration of particles at a surface of the beverage, by at least 1, 2, 3, 4, 5, 6, 7, 8, 9, 10, 15, 20, 25, 30, 35, 40, 45, 50, 60, 70, 80, 90, 100, 200, 300, 400, 500, 600, 700, 800, 900, 1000, 1100, 1200, 1300, 1400, 1500, 1600, 1700, 1800, 1900, or 2000 nephelometric turbidity units (NTU).

7. The dosage form according to claim 1, wherein the liquid beverage is water, a carbonated beverage, a malted beverage, wine, or liquor.

8. The dosage form according to claim 1, wherein the beverage is wine, and the visual indication includes cloudiness within the beverage and a film or foam at an upper surface of the beverage.

9. The dosage form according to claim 1, wherein the beverage is carbonated or malted, and the visual indication includes a foam at an upper surface of the beverage and residue from the dosage form on an inner surface of the vessel.

10. The dosage form according to claim 1, wherein the beverage is a liquor, and the visual indication includes dispersion of visible particles in the absence of a foam at a surface of the beverage.

11. The dosage form according to claim 1, wherein the beverage is water, and the visual indication includes dispersion of visible particles, a foam or film at a surface of the beverage, and at least a portion of the dosage form that is buoyant on the surface of the beverage.

12. (canceled)

13. The dosage form according to claim 1, wherein said dosage form produces the visual indication within the vessel when the dosage form has been crushed prior to being placed into the vessel.

14. The dosage form according to claim 1, wherein said dosage form produces the visual indication when placed in an intact state into the vessel containing the liquid beverage.

15. The dosage form according to claim 1, wherein the dosage form produces the visual indication regardless of whether the beverage is subjected to physical agitation after the dosage form is placed into the vessel.

16. The dosage form according to claim 1, wherein the dosage form produces the visual indication when the beverage is subjected to physical agitation after the dosage form is placed into the vessel.

17. (canceled)

18. The dosage form according to claim 1, further comprising an effervescent agent and wherein the effervescent agent assists with exposure of one or more of the excipients to the beverage.

19. (canceled)

20. The dosage form according to claim 18, wherein the effervescent agent is sodium carbonate, sodium bicarbonate, sodium phosphate, sodium hydrogen phosphate, sodium dihydrogen phosphate, sodium hydroxide, potassium carbonate, potassium bicarbonate, potassium hydrogen phosphate, potassium dihydrogen phosphate, potassium hydroxide, magnesium carbonate, magnesium hydroxide, magnesium oxide, calcium carbonate, calcium oxide, or any combination thereof.

21. The dosage form according to claim 1, further comprising a gelling polymer.

22. The dosage form according to claim 21, wherein said gelling polymer is a natural or synthetic starch, a natural or synthetic cellulose, an acrylate, a polyalkylene oxide, a polysaccharide, a polyacrylamide, an indene maleic anhydride polymer, a carbomer polymer, or any combination thereof.

23.-69. (canceled)

70. A method of treating depression in a subject comprising providing to the subject a dosage form of any one of claim 1.

71. A method of preventing drug-facilitated criminal exploitation of a subject comprising providing a dosage form according to claim 1.

72. A method of producing a visual indication of a dosage form's presence within a vessel containing a liquid beverage intended for ingestion by a subject that is a target of drug-facilitated criminal exploitation comprising providing a dosage form according to claim 1.
